(19) United States
(12) Patent Application Publication
(10) Pub. No.: US 2014/0308728 A1
(43) Pub. Date: Oct. 16, 2014

(54) NON-COVALENT BIOMOLECULE IMMOBILIZATION ON TITANIA NANOMATERIALS

(71) Applicant: The University of North Carolina at Chapel Hill, Chapel Hill, NC (US)

(72) Inventors: Jacob H. Forstater, Washington, DC (US); Alfred Kleinhammes, Chapel Hill, NC (US); Yue Wu, Chapel Hill, NC (US)

(73) Assignee: The University of North Carolina at Chapel Hill, Chapel Hill, NC (US)

(21) Appl. No.: 14/249,756

(22) Filed: Apr. 10, 2014

Related U.S. Application Data
(60) Provisional application No. 61/810,542, filed on Apr. 10, 2013.

(51) Int. Cl. C12N 11/14

(52) U.S. Cl. CPC C12N 11/14; Y10S 977/762; Y10S 977/962; B82Y 5/00
USPC 435/176; 423/610; 428/398; 428/402; 977/762; 977/962

(57) ABSTRACT

A biomolecule immobilization substrate comprising a titania nanotube is provided. Stable undercoordinated titanium sites on the surface of titanium dioxide nanotubes provide for the binding of biomolecules in multiple layers and aggregates. Corresponding methods of immobilizing and storing biomolecules are provided. Enzymatic or other biological activities of titania nanotube bound biomolecules can be preserved or enhanced.

### CROSS REFERENCE TO RELATED APPLICATION

This application claims the benefit of U.S. Provisional Patent Application Ser. No. 61/810,542, filed Apr. 10, 2013, the disclosure of which is incorporated herein by reference in its entirety.

### GRANT STATEMENT

This invention was made with government support under Grant No. DMR-0906547 awarded by the National Science Foundation. The government has certain rights in the invention.

### TECHNICAL FIELD

The presently disclosed subject matter relates to non-covalent biomolecule immobilization on titania nanomaterials. The presently disclosed subject matter also relates to biomolecule immobilization substrates and methods of using the same.

### BACKGROUND

Biomaterials, particularly protein based biomaterials, are a promising tool for creating robust highly selective biocatalysts. Assembled biomaterials for use as biocatalysts must sufficiently retain the near-native structure of proteins and provide molecular access to catalytically active sites. These requirements often exclude the use of conventional assembly techniques which rely on covalent cross-linking of proteins or entrapment within a scaffold.

Thus, a need remains for a means for creating stable protein-based biomaterials and other biomaterials and biocatalysts without the need for chemical modification. A need also remains for means and methods of immobilizing, storing and enhancing the properties of molecules, including biomaterials.

### SUMMARY

The presently disclosed subject matter provides biomolecule immobilization substrates as well as methods of immobilizing, storing, and enhancing the enzymatic activity of a biomolecules using titania nanomaterials. The presently disclosed subject matter provides mass spectrometry processes and methods for characterizing protein interactions and protein-drug interactions.

An object of the presently disclosed subject matter having been stated hereinabove, and which is achieved in whole or in part by the presently disclosed subject matter, other objects will become evident as the description proceeds when taken in connection with the accompanying Examples as best described hereinbelow.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIG. 1 is a schematic illustration of an exemplary structure of titania nanotubes (TiNT) as disclosed herein;

FIGS. 2A-2C are schematic illustrations of the surface structures of nanotubes (FIG. 2A), nanotiles (FIG. 2B) and nanoparticles (FIG. 2C).

FIGS. 3A through 3D are schematic illustrations of the process of multilayer adsorption and self-assembly of molecules, such as Ribonuclease A (RNaseA), for example, onto TiNT for a fixed TiNT concentration.

FIG. 4 is a data output illustrating an equilibrium adsorption isotherm of Ribonuclease A per unit surface area of TiNT. The dashed lines are drawn to guide the eye. The inset illustrates how sample composition was varied among trials;

FIGS. 5A and 5B are data outputs based on the self-assembly of RNaseA-TiNT aggregates as a function of the molar ratio, ξ. FIG. 5A is the adsorption isotherm showing the relative number of nanotube-bound (Δ) and unbound (free) (o) protein. In FIG. 5A units are moles protein normalized by total moles TiO₂. FIG. 5B includes the Dynamic Light Scattering (DLS) measurement showing that the mean aggregate diameter increases with ξ. The dashed line is drawn to indicate the critical aggregation concentration, ξ*;

FIGS. 6A through 6D are transmission electron microscopy (TEM) and scanning electron microscopy (SEM) images showing RNaseA protein adsorbed to TiNT. FIG. 6A is a TEM image of RNaseA-TiNT showing a 6-8 nm thick protein layer surrounding the nanotube. FIG. 6B is a SEM image at ξ<ξ* showing individual nanotubes coated with 6-8 nm of protein, indicating one to two layers of adsorbed protein. FIG. 6C is a TEM image at ξ>ξ* showing multiple nanotubes embedded in a large plaque suggesting the formation of large aggregates of multiple nanotubes. FIG. 6D is a SEM image at ξ>ξ* showing a large aggregate containing multiple protein-coated nanotubes;

FIGS. 7A and 7B are transmission electron microscopy (TEM) images showing Lysozyme protein adsorbed to TiNT, where multiple TiNT are shown surrounded by a larger protein plaque (FIG. 7A) which form micron-sized aggregates (FIG. 7B);

FIG. 8 is a bar plot of enzymatic activity (left axis) of RNaseA-TiNT samples normalized by the activity of the RNaseA control (line drawn at 100%), with reaction time constant (□) on the right axis (lines shown as a guide). Error bars show standard error; asterisks indicate statistical significant of relative enzymatic activities as compared to RNaseA control (*, p<0.05; ***, p<0.005; ****, p<0.0001);

FIG. 9 is an image of the results of electrophoresis (SDS-PAGE) of Ribonuclease A adsorbed on TiNT. All trials had the same TiNT concentration;

FIGS. 10A through 10F are SEM images of different TiO₂ nanomaterials (FIGS. 10A (nanoparticles), 10B (nanotiles) and 10C (nanotubes)) and structures formed after interacting with Ribonuclease A (FIGS. 10D (nanoparticles), 10E (nanotiles) and 10F (nanotubes));

FIG. 11 is a data output of thermogravimetric analysis (TGA) measurements of TiNT (dashed line), anatase nanoparticles (TiNP; dashed and dotted line), and anatase (001) nanotiles (NTile; dotted line). The inset provides a close up view of NTile and TiNP weight loss curves;

FIGS. 12A and 12B are schematic illustrations of the interaction of TiNT in an oil-water interface. FIG. 12A is a schematic illustration of a TiNT-stabilized water-in-oil Pickering emulsion. FIG. 12B illustrates a particle of radius R, at the oil-water interface, with interfacial tension, γ, shown between the particle (p), oil (o) and water (w) phases; and

FIGS. 13A and 13B are data output of equilibrium adsorption isotherm Ribonuclease A, Lysozyme, and Ubiquitin on TiNT, as determined by a fluorometric assay. In FIG. 13A the surface coverage (y-axis) is expressed in terms of the number of protein adsorbed per unit area nanotube, and the equilibrium concentration (x-axis) of each protein is expressed in μM. In FIG. 13B, the log-lin plot of lower equilibrium concentrations highlights the different adsorption isotherms.

### DETAILED DESCRIPTION

Provided herein are biomolecule immobilization substrates, apparatuses, devices, packages, components, applications and/or methods of immobilizing, storing and/or enhancing one or more properties of molecules, including biomaterials. In some embodiments, provided herein are biomolecule immobilization substrates and/or methods comprising a titania nanotube, wherein the titania nanotube can comprise a surface and stable undercoordinated titanium sites on the surface.

In some embodiments, substrates and methods are provided for creating stable protein-based biomaterials and other biomaterials and biocatalysts without the need for chemical modification. Such substrates and methods can in some embodiments provide for means of immobilizing, storing and enhancing the properties of molecules, including biomaterials.

More particularly, in some embodiments, titania nanotubes (TiNT) are provided that can initiate and template the self-assembly of biomolecules, such as enzymes, while maintaining their biological activity, e.g. catalytic activity. As discussed in more detail herein below, initially the biomolecules can form multilayer thick ellipsoidal aggregates centered on a nanotube surface, and subsequently these nanosized entities can assemble into a micron-sized aggregates. This surprising phenomenon is uniquely associated with the active anatase-(001) like surface of TiNT and does not occur on other anatase nanomaterials, which contain significantly fewer undercoordinated titania (Ti) surface sites. Where the biomolecules are enzymes the aggregates can have enhanced enzymatic activity and contain as little as 0.1 wt % TiNT.

Thus, in some embodiments, disclosed herein are nanotechnology-enabled mechanisms, substrates and/or methods of biomaterial or biomolecule growth that provides new routes for creating stable protein-based and other biomaterials and biocatalysts without the need for chemical modification.

In some aspects, provided herein are biomolecule immobilization substrates and/or methods comprising a titania nanotube, wherein the titania nanotube can in some embodiments comprise a surface and stable undercoordinated titanium sites on the surface, wherein the titania nanotube binds biomolecules. Accordingly, in some embodiments a method of immobilizing a biomolecule is provided, comprising: providing a titania nanotube comprising a surface and stable undercoordinated titanium sites on the surface, providing a biomolecule to be immobilized, and exposing the biomolecule to the titania nanotube, whereby the biomolecule is immobilized on the surface of the titania nanotube. Additionally, in some aspects, a method for storing a biomolecule is provided, comprising providing a titania nanotube comprising a surface and stable undercoordinated titanium sites on the surface, providing a biomolecule to be stored, and mixing the biomolecule and the titania nanotube in a solution, whereby the biomolecule is immobilized on the surface of the titania nanotube and is stable for storage. In some embodiments, a method for enhancing the enzymatic activity of a biomolecule is provided, comprising: providing a titania nanotube comprising a surface and stable undercoordinated titanium sites on the surface, providing a biomolecule with enzymatic activity, and mixing the biomolecule and the titania nanotube in a solution, whereby the biomolecule non-covalently binds to the titania nanotube, whereby the enzymatic activity of the biomolecule is increased above that of a biomolecule with enzymatic activity that is not bound to a titania nanotube.

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a” and “an” mean “one or more” when used in this application, including the claims.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including,” “containing,” or “characterized by” is inclusive or open-ended and does not exclude additional, unrecited elements or method steps. “Comprising” is a term of art used in claim language which means that the named elements are present, but other elements can be added and still form a construct or method within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed as a “p value”. Those p values that fall below a user-defined cutoff point are regarded as significant. In some embodiments, a p value less than or equal to 0.05, in some embodiments less than 0.01, in some embodiments less than 0.005, and in some embodiments less than 0.001, are regarded as significant. Accordingly, a p value greater than or equal to 0.05 is considered not significant.

As used herein the terms “biomolecule”, “biomaterial”, “biocompound” and “biogenic substance” are used interchangeably and refer any molecule or compound that is produced by or associated with a living organism. By way of example and not limitation, a biomolecule can comprise macromolecules such as proteins, polysaccharides, lipids, and nucleic acids, as well as small molecules such as primary metabolites, secondary metabolites, and natural products. Further examples of biomolecules include proteins, enzymes, antibody, antigen, hapten, lipids, polysaccharides, carbohydrates, glycolipids, phospholipids, sterols, glycerolipids, glycerides, vitamins, hormones, neurotransmitters, metabolites, secondary metabolites, monomers, oligomers, polymers, biomonomer, bio-oligomers, biopolymers, amino acids, peptides, oligopeptides, polypeptides, monosaccharides, oligosaccharides, nucleosides, nucleotides, oligonucleotides, polynucleotides, nucleic acids (DNA, RNA, PNA), gene, chromosome, aptamer and lignin.

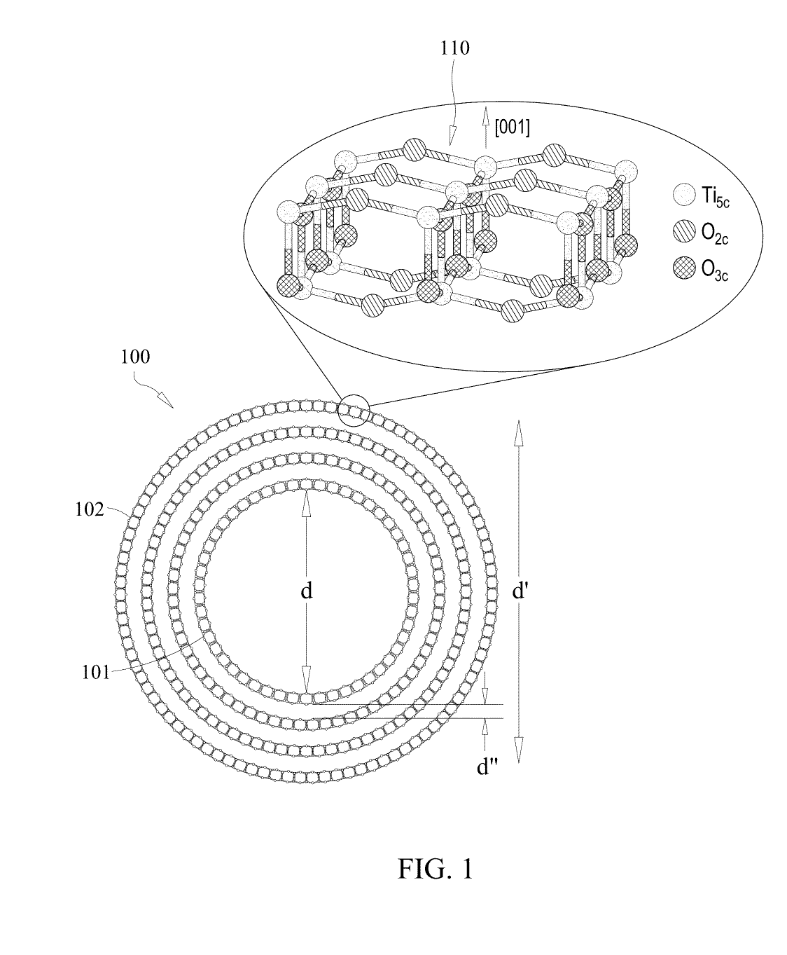

Turning now to the Figures, FIG. 1 is a schematic illustration of an exemplary structure of a titania nanotube (TiNT) 100 as disclosed herein when viewed from the top down. In some embodiments TiNT 100 can comprise a series or plurality of conical or tubular nanostructures, one fitting inside the other, with an inner most 101 and out most 102 nanotube. For illustrative purposes only, FIG. 1 depicts a TiNT comprising 4 tubular structures with an inner most 101 and out most 102 tubular nanostructures. In some embodiments, TiNT and other nanotubes can have between 3 to 6 layers or tubular nanostructures. In some embodiments, TiNT 100 can have an inner diameter d (or diameter of the inner most 101 nanostructures) of about 5 nm to about 7 nm. In some embodiments, TiNT 100 can have an inner diameter d of about 6 nm. In some embodiments, TiNT 100 can have an outer diameter d′ (or diameter of the outer most 102 nanostructures) of about 8 nm to about 14 nm. In some embodiments, TiNT 100 can have an outer diameter d′ of about 14 nm. In some embodiments, TiNT 100 can have an interlayer spacing d″ of about 0.87 nm. In some embodiments, TiNT 100 can have a length of about 50 nm to about 3,000 nm. Finally, in some aspects, TiNT 100 can have an isoelectric point of about 2.0 pH to about 3.0 pH.

To better illustrate the detail of the structure of TiNT 100, a portion of the outer surface 102 of TiNT 100 is shown in an amplified view in FIG. 1. TiNT 100 as disclosed herein can have an outer surface 102 with an exposed anatase (001)-like surface 110. The exposed anatase (001)-like surface 110 can in some embodiments comprise undercoordinated Titanium Ti_{5c} and can be stable against hydroxylation. In some embodiments, both 2-coordinated oxygen O_{2c} and 3-coordinated oxygen O_{3c} can be present. A TiNT dispersion can in some aspects be stable at physiological pH.

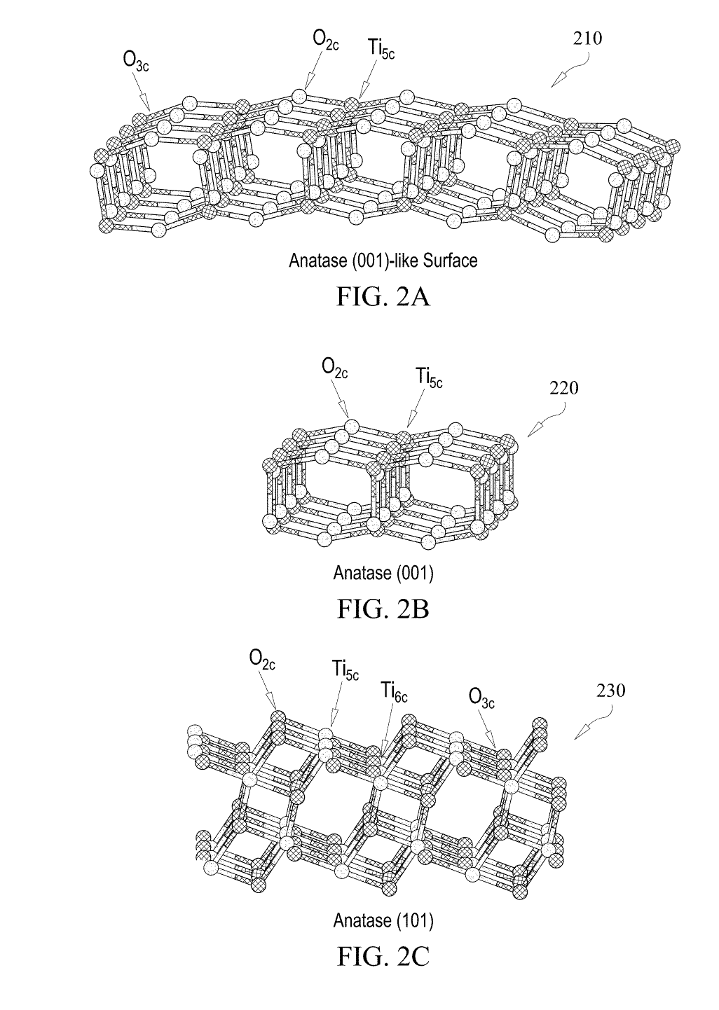

Similar to FIG. 1, FIG. 2A depicts an exposed surface 210 of TiNT 100. As seen in FIG. 2A, the exposed surface 210 of TiNT 100 has an anatase (001) 220 structure (FIG. 2B), or is anatase (001)-like, versus an anatase (101) 230 structure (FIG. 2C). Such a surface can be formed by delaminating anatase along the [001] direction and curving the delaminated anatase (001) surface 210 around the [010] axis. This can be formed by cleaving the anatase unit cell through its apical bonds, along the [001] direction, at 0.65 of the unit cell height. The unit cell can then be stacked along the [001] direction with an interlayer spacing of 0.87 nm, with each layer shifted by half a unit cell in the [100] and [010] directions, resulting in a loss of registry between adjacent layers and agreeing with experimentally observed glide shift. The surface Ti sites on clean bulk (001) surface are all fivefold coordinated and under ambient conditions these sites are hydroxylated by dissociative water adsorption. In contrast, water is only molecularly adsorbed on the surface of the nanotube, which also contains only fivefold coordinated Ti_{5c} sites. The stability of these groups against hydroxylation leaves these groups open to react and can be crucial to its reactivity.

Referring again to FIGS. 1 and 2A, immobilized transition metals such as titanium can in some embodiments interact with amino acids. Such non-covalent interactions between transition metal ions and protein surface residues can in some aspects modify protein-protein interfacial interactions. Using TiNT 100, disclosed herein is the first successful non-covalent assembly of enzymes into an insoluble solid that contains over 99% enzymes by weight and has enhanced catalytic activity. This is achieved by a disclosed enzyme assembly mechanism enabled by the unique, undercoordinated, surface chemistry of the TiO₂ anatase (001) surface 110 or 210 on TiNT 100 as depicted in FIGS. 1 and 2A. After introducing an extremely low concentration of TiNT 100, which have an active anatase-(001)-like surface, into an enzyme solution, the growth of multilayer enzyme coatings was observed on the TiNT, followed by assembly of such enzyme-coated objects into large micron-sized structures. As demonstrated herein, and without being bound by any particular theory or mechanism of action, the TiNT's stable undercoordinated Ti sites Ti_{5c} can in some aspects be required for this phenomenon.

As detailed further herein, in some embodiments an enzyme monolayer adsorbs to the nanotube surface, interacting with the undercoordinated Ti sites of the anatase (001) surface. This monolayer can then act as a seed for the further growth of more than 50 layers of enzyme. Finally these enzyme-coated nanotubes can in some aspects reach a critical size, and self-assemble along with additional free enzyme, forming larger micron-sized structures. The instant disclosure is the first report of non-covalent immobilization of extensive protein multilayers on a nanomaterial and the first report of the emergence of a self-assembled mesophase of protein-nanotube conjugates. These findings present a nanotechnology-enabled mechanism of biomaterial growth and open a new route for creating stable protein-based materials, in particular, enzyme-based biocatalysts.

As evinced by the Examples herein below, the aggregation of biomolecules on TiNT requires more than just the presence of the anatase (001) surface 110. The stability of the surface Ti groups Ti_{5c} against hydroxylation is a factor in such aggregation. The disclosed TiNT 100, and methods of making the same, comprise exposed and stable undercoordinated Ti surface sites. The bond strain induced by the nanotube's curvature (see, e.g. FIGS. 1 and 2A) can prevent hydroxylation of the nanotube surface 110 or 210. When this is removed the undercoordinated Ti groups can be instantly hydroxylated and no longer available to react. These findings suggest that the exposed, stable, undercoordinated Ti sites on the TiNT surface is involved in initiating the self association of the free and bound biomolecules, e.g. proteins.

Thus, in some embodiments biomolecule immobilization substrates comprising a titania nanotube are provided. Such a substrate can in some embodiments comprise a titania nanotube with a surface having stable undercoordinated titanium sites thereon.

In some embodiments, substrates and methods are provided for creating stable protein-based biomaterials and other biomaterials and biocatalysts without the need for chemical modification. Such substrates and methods can in some embodiments provide for means of immobilizing, storing and enhancing the properties of molecules, including biomaterials.

More particularly, in some embodiments, titania nanotubes (TiNT) are provided that can initiate and template the self-assembly of biomolecules, such as enzymes, while maintaining their biological activity, e.g. catalytic activity. As discussed in more detail herein below, initially the biomolecules can form multilayer thick ellipsoidal aggregates centered on a nanotube surface, and subsequently these nanosized entities can assemble into a micron-sized aggregates. This surprising phenomenon is uniquely associated with the active anatase-(001) like surface of TiNT and does not occur on other anatase nanomaterials, which contain significantly fewer undercoordinated titanium (Ti) surface sites. Where the biomolecules are enzymes the aggregates can have enhanced enzymatic activity and contain as little as 0.1 wt % TiNT.

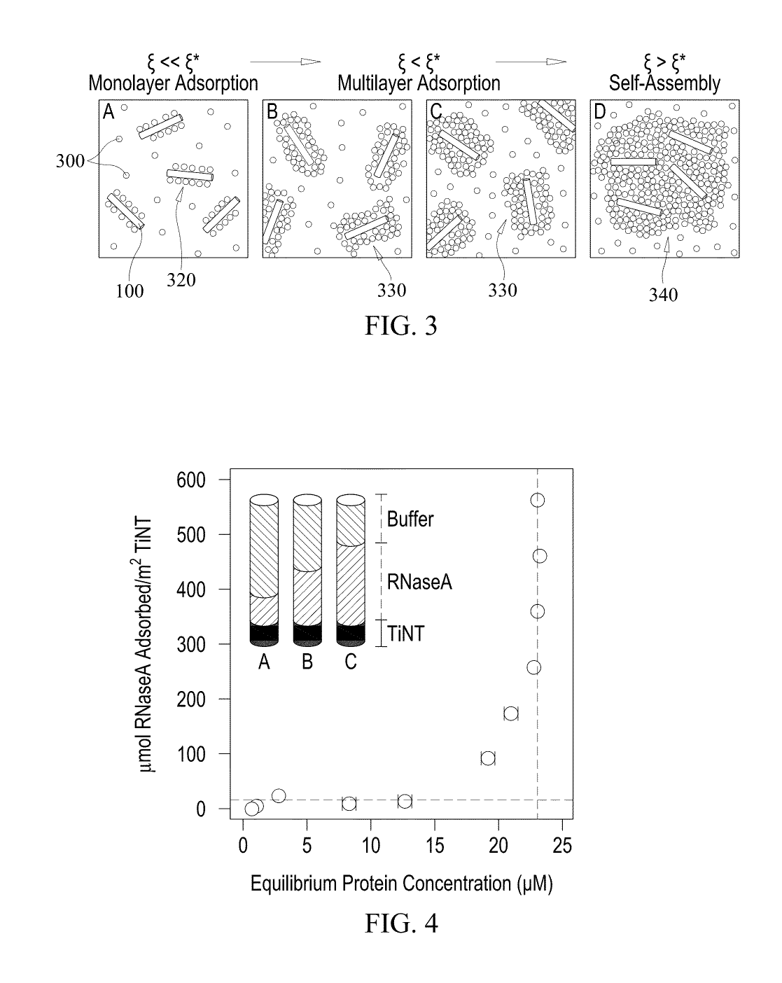

Without being bound by any particular theory or mechanism of action, in some embodiments a model for the observed interactions between biomolecules and TiNT is illustrated in FIG. 3. Initially, the system consists of biomolecules 300, e.g. monomeric protein, and individual nanotubes (TiNT) 100 coated with biomolecules 300. At extremely low biomolecule concentrations (ξ<<ξ*, where is the molar ratio of biomolecule to TiNT, and ξ* is the critical aggregation concentration), biomolecules 300 adsorb as monolayers 320 (FIG. 3A). As the biomolecule 300 concentration is increased (ξ<ξ*), extensive biomolecule multilayers 330 form on the nanotubes 100 (FIGS. 3B and 3C), continuing until a critical concentration of free biomolecule 300 is reached. Above the critical concentration excess biomolecules 300 and the dispersed individual biomolecule-nanotube conjugates assemble into an aggregate mesophase 340 consisting of large, prolate ellipsoidal structures that can contain multiple nanotubes 100 and biomolecules 300 (FIG. 3D).

This adsorption behavior is an indication of self-assembly. Other phenomenon involving the emergence of an aggregate mesophase is observed in the formation of supramolecular assemblies and in other self-assembling systems such as liposomes or giant vesicles.^{2,35} Thermodynamically, the structural transition between the dispersed (FIG. 3C) and self-assembled state (FIG. 3D) is favorable only if assembly reduces the Gibb's free energy of the system. The critical aggregation concentration (CAC) at which this transition occurs is determined by the chemical potential difference of any two phases in the system. At the critical transition, the packing limits for the biomolecule, e.g. protein, on the nanotube surface have been reached. This forces the system to rearrange and reassemble through interaggregate interactions which reduce the Gibbs free energy. The inhomogeneous microstructure and prolate shape of the observed aggregates are characteristic of binary supraself-assembled systems. The interpenetrating packing of the multilayer-coated nanotubes allows for a higher packing volume fraction, while the prolate shape decreases the Gaussian curvature and reduces the interfacial tension.

Results provided herein demonstrate that that the process of aggregating or immobilizing biomolecules on TiNT can be exploited to create biomolecule-TiNT conjugates and/or aggregates that in some aspects can retain a biological or other function native to the biomolecule prior to aggregation. For example, in some aspects proteins or enzymes can be immobilized on TiNT to form protein-TiNT conjugates and/or aggregates that form functional, insoluble enzyme biocatalysts. The enzymatic activity of the multilayer and self-assembled protein/enzyme-nanotube conjugates can be equal to or substantially similar to that of non-conjugated or wild-type versions of the same enzyme. For example, in some embodiments, the enzymatic activity of an enzyme-nanotube conjugate can be about 50% to 100% of the enzymatic activity of a non-conjugated version of the same enzyme, or about 75% to 100% of the enzymatic activity of a non-conjugated version of the same enzyme. In some embodiments, the enzymatic activity of an enzyme-nanotube conjugate can be about 99%, 98%, 97%, 96%, 95%, 90%, 80%, 70%, 60% or 50% of the enzymatic activity of a non-conjugated version of the same enzyme. In some embodiments, the enzymatic activity of an enzyme-nanotube conjugate can be enhanced such that it is greater than the enzymatic activity of a non-conjugated version of the same enzyme. For example, in some embodiments, the enzymatic activity of an enzyme-nanotube conjugate can be about 101% to about 150% of the enzymatic activity of a non-conjugated version of the same enzyme, or about 101% to 125% of the enzymatic activity of a non-conjugated version of the same enzyme. In some embodiments, the enzymatic activity of an enzyme-nanotube conjugate can be about 101%, 102%, 103%, 104%, 105%, 110%, 120%, 130%, 140% or 150% of the enzymatic activity of a non-conjugated version of the same enzyme.

Additionally, in some embodiments, adhering a protein or enzyme to a TiNT as disclosed herein can increase the active lifetime of adsorbed enzymes or increase the accessibility of adsorbed enzymes by forming a more ordered or less tortuous assembly. Relative activity is a function of the active lifetime of an enzyme and accessibility, while the reaction kinetics are an indicator of the diffusional resistance the substrate experiences. A more porous, or less tortuous immobilized layer, can in some embodiment increase the enzyme accessibility and substrate diffusivity, thereby resulting in enhanced activity as compared to a wild-type or bulk protein. Thus, in some embodiments self-assembly of an enzyme on a TiNT can alter the microstructure of the immobilized multilayers, forming either a more porous or less tortuous network of immobilized proteins or enzymes than is found in the multilayer state. As increases, more protein is immobilized and the number of proteins residing on the exterior of the self-assembled aggregates can also increase. As the aggregate surface area increases, reactions more frequently occur on the surface and more collisions between substrate and the enzyme occur, shifting the reaction kinetics from diffusion limited to reactant limited regime.

Thus, provided herein are titania nanotubes with a high density of unterminated undercoordinated Ti surface sites that are able to immobilize extraordinarily large quantities of biomolecules, in some instances over 1,000 times above monolayer coverage. This unexpected result is in contrast to other forms of TiO₂ nanomaterials that do not show such properties. This phenomenon has not been reported previously with any other nanomaterial. Biomolecule immobilization and assembly on titania nanotubes occurs in two different stages. First, at low biomolecule-to-TiO₂ molar ratios, biomolecule immobilization takes place up to about 55 layers of coverage, in the case of RNaseA. The coverage then remains constant until a critical biomolecule-to-TiO₂ molar ratio is reached. Upon reaching this critical ratio, the system self assembles into large aggregates, above which any subsequently added biomolecules incorporate into the existing self-assembled aggregates. Such self-assembled products can be micron-sized, immobilizing as much as 1,000 g/g protein/TiO₂. Moreover, such self-assembled aggregates can in some aspects completely retain or even enhance biological activity, e.g. enzymatic activity.

In some embodiments, disclosed herein are nanotechnology-enabled mechanisms, substrates and/or methods of biomaterial or biomolecule growth that provides new routes for creating stable protein-based and other biomaterials and biocatalysts without the need for chemical modification.

In some aspects, provided herein is a biomolecule immobilization substrate comprising a titania nanotube, wherein the titania nanotube comprises a surface and stable undercoordinated titanium sites on the surface, wherein the titania nanotube binds biomolecules. In some aspects, the stable undercoordinated titanium sites on the surface of the titania nanotube can bind biomolecules under physiological conditions. Such binding of biomolecules can be non-covalent. As defined herein, a biomolecule, also referred to as biomaterial, biocompound and/or biogenic substance, can comprise any molecule or compound that is produced by or associated with a living organism. A list of exemplary biomolecules in provided hereinabove. In some aspects, the biomolecule can comprise an enzyme. An enzyme that is bound to a substrate can have an enzymatic activity substantially similar to an enzymatic activity of an unbound enzyme. Indeed, as the evidence herein shows, in some aspects the bound enzyme can have an enzymatic activity that is increased as compared to an enzymatic activity of an unbound enzyme. Such an immobilization substrate can bind biomolecules to the surface of the titania nanotube in multiple layers.

Additionally, in some embodiments a method of immobilizing a biomolecule is provided, comprising: providing a titania nanotube comprising a surface and stable undercoordinated titanium sites on the surface, providing a biomolecule to be immobilized, and exposing the biomolecule to the titania nanotube, whereby the biomolecule is immobilized on the surface of the titania nanotube. Exposing the biomolecule to the titania nanotube can comprise combining a biomolecule and a titania nanotube into a solution. The stable undercoordinated titanium sites on the surface of the titania nanotube can bind biomolecules under physiological conditions. In some instances, the binding of biomolecules is non-covalent. As discussed herein, the biomolecule, also referred to as biomaterial, biocompound and/or biogenic substance, can comprise any molecule or compound that is produced by or associated with a living organism. A list of exemplary biomolecules in provided hereinabove. In some aspects, the protein can be an enzyme. An enzyme that is bound to a substrate can have an enzymatic activity substantially similar to an enzymatic activity of an unbound enzyme. Indeed, as the evidence herein shows, in some aspects the bound enzyme can have an enzymatic activity that is increased as compared to an enzymatic activity of an unbound enzyme. Such an immobilization substrate can bind biomolecules to the surface of the titania nanotube in multiple layers.

In some embodiments such a method of immobilizing a biomolecule can further comprise exposing a plurality of biomolecules to the titania nanotube. The biomolecules can bind to the surface of the titania nanotube in multiple layers. In some aspects, exposing a plurality of biomolecules to the titania nanotube can result in the self-organized formation of biomolecule-nanotube conjugates. In the above methods of immobilizing a biomolecule the bound biomolecules can substantially maintain their original confirmation.

In some embodiments, a method for storing a biomolecule is provided, comprising providing a titania nanotube comprising a surface and stable undercoordinated titanium sites on the surface, providing a biomolecule to be stored, and mixing the biomolecule and the titania nanotube in a solution, whereby the biomolecule is immobilized on the surface of the titania nanotube and is stable for storage. In some aspects, a plurality of biomolecules can be mixed with a plurality of titania nanotubes. More particularly, a quantity of biomolecules can be mixed with a quantity of titania nanotubes sufficient to form a monolayer of biomolecules on the nanotubes. Likewise, a quantity of biomolecules can be mixed with a quantity of titania nanotubes sufficient to form a multilayer of biomolecules on the nanotubes. And in some embodiments, a quantity of biomolecules can be mixed with a quantity of titania nanotubes which results in the self-organized formation of biomolecule-nanotube aggregates. Such biomolecule-nanotube aggregates are about one micron in size.

In some embodiments, a method for enhancing the enzymatic activity of a biomolecule is provided, comprising: providing a titania nanotube comprising a surface and stable undercoordinated titanium sites on the surface, providing a biomolecule with enzymatic activity, and mixing the biomolecule and the titania nanotube in a solution, whereby the biomolecule non-covalently binds to the titania nanotube, whereby the enzymatic activity of the biomolecule is increased above that of a biomolecule with enzymatic activity that is not bound to a titania nanotube. In some aspects, a plurality of biomolecules can be mixed with a plurality of titania nanotubes. More particularly, a quantity of biomolecules can be mixed with a quantity of titania nanotubes sufficient to form a monolayer of biomolecules on the nanotubes. Likewise, a quantity of biomolecules can be mixed with a quantity of titania nanotubes sufficient to form a multilayer of biomolecules on the nanotubes. And in some embodiments, a quantity of biomolecules can be mixed with a quantity of titania nanotubes which results in the self-organized formation of biomolecule-nanotube aggregates. Such biomolecule-nanotube aggregates are about one micron in size. Finally, the enzymatic activity of the biomolecules bound to the titania nanotubes can be increased by about 10% to about 90% as compared to an unbound enzyme.

In some embodiments TiNT of the instant disclosure can be produced or synthesized by starting with titania nanotubes that are hydrothermally synthesized and shortened as previously described (Mogilevsky et al., 2008(b); Chen et al., 2009). Particularly, anatase nanoparticles can be added to freshly prepared 10 M NaOH. The mixture can then be sealed in a PTFE-lined stainless steel autoclave and maintained at about 135° C. for about 72 hr. The resulting material can then be repeatedly washed with distilled water and HCl (0.1 M) until the supernatant reaches a pH of about 5 to about 6. Subsequently, the nanotubes can be shortened by wet ball milling in a laboratory ball mill (Glen-Mills, Clifton, N.J., United States of America), also referred to as cryomilling. In some aspects, either the nanotubes were used in the suspended form as described or the suspension of long-nanotubes was further concentrated by pelleting the nanotubes in a centrifuge and removing the clear supernatant solution. In some embodiments, particularly in a small-batch synthesis, cryomilling can comprise mixing approximately 50 mL of the concentrated nanotube suspension with about 30 g of 100 um diameter ZrO2 beads (Glen-Mills) and placing the suspension in a grinding vessel. The grinding vessel can be surrounded with a cooling ice bath and placed inside a refrigerated room held at 4° C. and the suspension can then be ground for up to 45 minutes. Following ball milling, the supernatant, which contains only shortened nanotubes, can be decanted and centrifuged to remove any excess grinding media. Additional cryomilled nanotubes can in some embodiments be recovered by washing the grinding media off with water.

Methods of producing TiNT as disclosed herein, and particularly that which provides TiNT with the ability to immobilize large quantities of biomolecules, differs from previous titania nanotube synthetic methods. In particular, in some embodiments the disclosed methods of synthesizing TiNT can comprise a cryomilling procedure in addition to or in place of previous titania nanotube shortening procedures. Without being bound by any particular theory or mechanism of action, the disclosed cryomilling procedure can provide, at least in part, for the disclosed TiNT with a surface having stable undercoordinated titanium sites capable of binding significant quantities of biomolecules. In some aspects, the disclosed cryomilling procedure of synthesizing TiNT can utilize an increased amount, e.g. 100-2000 mg, of nanotubes as compared to previous methods (about 10-20× more). In some aspects, the disclosed cryomilling procedure of synthesizing TiNT can utilize a larger amount of grinding media, e.g. 30-60 grams, as compared to previous methods. In some aspects, the disclosed cryomilling procedure of synthesizing TiNT is performed at lower temperatures, e.g. in an ice-water bath in a 4° C. cold room. In some aspects, the grinding time can differ from previous methods, and can in some embodiments include a continuous grinding as opposed to intermittent grinding. Additionally, in some aspects, the grinding time can be significantly less, e.g. about 20 min to about 45 min. In some aspects, the disclosed cryomilling procedure does not require centrifugation to remove grinding media. Instead, the grinding media can fall out/sediment naturally (occurs in a few minutes or less normally). Importantly, the finally concentration of produced TiNT can be significantly enhanced, in some embodiments to about 200 to about 400 mg/mL, which is about 200 to 400 times higher concentration than previous methods. Finally, in some embodiments, additional recovery of TiNT product from grinding media can be achieved, if desired, using the disclosed cryomilling technique. For example, grinding media can be rinsed with water in a container, allowed to rest briefly to allow a milky solution to rise to the top which can then be decanted (this contains significant concentration of about 20-30 mg/mL of additional cryomilled nanotubes). In some aspects, cryomilling provides the ability to produce highly concentrated, stable dispersions of nanotube or nanotube-like objects. The yield, suspendability, and stability can in some instances be significantly improved as compared to methods of synthesizing titania nanotubes without cryomilling.

Such processes of synthesizing TiNT can in some embodiments be scaled-up to include larger volumes, different grinding media, employ different operational conditions without departing from the scope of the instant disclosure. By way of example and not limitation, the grinding media can in some embodiments be made of a variety of ceramics, metals, and related materials. For example, ceramics can include, but are not limited to, aluminum oxide, fused zirconium oxide, sintered zirconium oxide, sintered zirocnium silicate and can employ a variety of additional stabilizing additives, such as Y₂O₃, CeO, Al₂O₃, MgO, or other additives well understood to those experienced in the art. Other materials could include, but are not limited to, tungsten carbide and various alloys of steel.

In addition to the methods of immobilizing, storing, and enhancing the enzymatic activity of a biomolecules using titania nanomaterials as discussed above, TiNT of the presently disclosed subject matter can in some embodiments have a plurality of other uses and applications. In particular, such embodiments can utilize the unique surface chemistry of the disclosed TiNT to improve upon existing technologies.

In some embodiments, TiNT as disclosed herein can be used in pickering emulsions for various applications. By way of example and not limitation, TiNT can have numerous applications in petrophysical applications, including use as a dielectric contrast agent. Such an application is possible due to the properties of cryomilled TiNT that allow them to migrate to or aggregate at an oil-water interface. See, e.g., Example 10 below.

To elaborate, in some embodiments TiNT can form stable oil/water or water/oil emulsions for use in enhanced oil recovery or environmental remediation/cleanup. Such methods can offer improvements over existing colloidal particles used for such purposes, and can improve stability of oil/water emulsions over a wider range of pH, salt, and temperature ranges. These benefits can include, for example: the ability to recover and transport oil over longer distances, such as between injection to production wells and within the oil reservoir. As compared to existing, larger, colloidal particles, TiNT can in some embodiments migrate through smaller pores (pore throats) in geological structures, enabling longer range enhanced oil recovery. TiNT stabilized oil/water emulsions or water/oil emulsions can be used to transport viscous oil through pipelines with smaller pressure drops and displace viscous oil from high permeability rocks.

In some aspects, TiNT can be used as an additive within enhanced oil recovery and drilling applications to alter fluid properties or fluid-rock interactions. Within injected fluids, TiNT can be used to alter suspension rheology and enhance viscosity, density, alter surface tension, improve emulsification, and alter the thermal properties of the fluid. The fluid-rock interactions can be modified by using TiNT as an additive to alter the wettability and heat transfer coefficient. In some embodiments nanofluids can be used. In such instances wettability can be achieved by adsorption of the TiNT onto the rock, occurring to do disjoining pressure. As an additive it can be used to alter surface wettability in water-injection applications used in enhanced oil recovery and within aqueous drilling fluids. Alternatively, in some aspects nanoemulsions can be used. Nanoemulsions can be small enough to pass through pore throat in reservoir rock without significant retention. Finally, some embodiments can comprise nanofoam formation. In such instances, TiNT can be an additive to water during CO₂ flooding. TiNT can improve sweep efficiency by stabilizing viscous fingering and flow through permeable zones. This can significantly improve the lifetime and stability over a wide range of pH, salt, and temperature.

In some aspects, TiNT can be used on its own or integrated with polymer to form membranes for gas separations, asphaltene removal, or to remove harmful or aggregates substances. By way of example and not limitation, polyimide/TiNT membranes can be used in separations.

In some embodiments, devices, systems and/or methods can be used form surface patterning of nanotubes on a substrate. For example, in some embodiments, nanotubes, such as for example TiNT as disclosed herein, can be deposited and patterned on a substrate by a number of techniques familiar to one of ordinary skill in the art. In some aspects, a pattern of silicon substrate can be used with a silane containing a phosphonated headgroup or an exposed endiol ligand. These groups can for example be patterned on the surface using conventional lithographic techniques or by employing a stamp to place the silane groups on the surface. Owing to the high affinity of nanotubes for these groups, TiNT can then be applied to these exposed groups on a surface to thereby bind the TiNT to thereby resulting in a patterned surface of TiNT.

In some embodiments the majority of the nanotube surface area can still be available to interact with biomolecules even after being affixed to a substrate in a desired pattern or location. This substrate or nanotube patterned device or apparatus can then be used to immobilize proteins or biomolecules based on the affinities of the TiNT toward the proteins or biomolecules. By way of example and not limitation, the TiNT can comprises a surface and stable undercoordinated titanium sites on the surface which as disclosed herein provide for the ability to bind, in some instances significant quantities, of proteins or biomolecules.

Once the nanotubes are patterned on the surface, electrodes can in some embodiments be deposited or applied using existing techniques. A nanotube, e.g. TiNT, patterned substrate can then be exposed to one or more proteins or biomolecules of interest. Such a device or apparatus can be used as a sensor, switch and/or detector capable of sensing or detecting one or more proteins or biomolecules based on the patterning of the nanotubes.

Nanotubes as disclosed herein, such as for example TiNT, can be used as chemical or biological sensors or in the creation of devices or systems acting as chemical or biological sensors. In some embodiments, the intrinsic properties of the nanotube, such as for example impedance, capacitance, or resonant frequency, can be altered by protein immobilization, self-assembly, or other aggregation on the nanotube as disclosed herein. Measuring changes in these or other intrinsic properties based on the immobilization, self-assembly, or other aggregation of proteins and/or biomolecules can be used as a means for detecting or sensing the presence of a particular protein and/or biomolecule. Such a biosensor can in some embodiments be used for the detection or classification of proteins immobilized on a substrate or for monitoring the kinetics of any of these processes.

In some embodiments, such a sensing device or system can comprise TiNT immobilized on a substrate (as described hereinabove in the section Patterned Nanotube Devices) and electrodes deposited thereon. Alternatively, in some embodiments cyclic voltammetry can be used to examine proteins which undergo redox reactions or other electrochemically detected reactions on a surface or a biosensor with TiNT as disclosed herein. By way of example and not limitation, such a biosensor can comprise a glucose sensor, which can employ glucose oxidase.

In some embodiments, materials and methods are provided for using monolithic and/or composite materials containing titanium dioxide, or other nanotubes as disclosed herein, e.g. TiNT, for use as chromatographic materials. Such chromatographic materials can be useful in recovering a target compound, such as a peptide or protein, from an aqueous medium. In particular, in some embodiments such monolithic materials can comprise TiNT as well as related forms while composite materials can comprise TiNT distributed within a polymer network or embedded in a polymer material. In some embodiments, such materials can have improved mechanical or chemical properties in addition to a pore geometry or orientational alignment capable of selective chromatographic separation. By way of example and not limitation, TiNT can comprises a surface and stable undercoordinated titanium sites on the surface which as disclosed herein provide for the ability to bind, in some instances significant quantities of proteins or biomolecules.

In some aspects, such materials can be useful in commercial applications such as for example packings for chromatography columns, chromatographic cartridges, chromatographic plates, sequestering reagents, specialized biomolecule separation kits, solid supports for combinatorial chemistry, solid supports for oligosaccharide and/or polypeptide and/or oligonucleotide synthesis, solid supports for biological and/or chemical assays, solid supports for transport and/or storage of biomolecules, solid supports for biomolecule immobilization and/or capture, solid supports for processing and/or purification of molecules, catalyst supports, filtration membranes, microtiter plates, scavenger resins, solid phase organic synthesis supports, solid phase extraction devices, and packing materials for microchip separation and/or processing devices. In some embodiments, nanotubes, e.g. TiNT, can be used to fractionate, purify, capture, or separate a component molecule or substance from a gas, solution, dispersion, or suspension containing one, two, or a plurality of components. For example, a nanotube can participate in a variety of chromatographic processes as a resin, sorbent, or other chromatographic media. For example, the nanotubes as a monolith or composite material can be used for Affinity Chromatography, Metal-Chelate Chromatography, Ion Exchange Chromatography, Size Exclusion Chromatography, Expanded Bed Adsorption Chromatography, Solid-Phase Extraction, Reversed-Phase Chromatography, Normal-Phase Chromatography, Displacement Chromatography, Aqueous Normal-Phase Chromatography, and/or Capillary Electrochromatography.

In some aspects, devices containing the nanotubes for chromatographic applications can be provided. By way of example and not limitation, TiNT as disclosed herein can comprises a surface and stable undercoordinated titanium sites on the surface which provide for the ability to bind, in some instances significant quantities, proteins or biomolecules. Nanotubes as disclosed herein can in some embodiments be used as a resin within a column or fixed bed, or incorporated within or as part of another material, for chromatographic separations.

Nanotubes can be used as either a stationary phase or as a mobile phase. Additionally, nanotubes can be incorporated into other chromatographic resins or packed into a column with a porous frit or membrane at the top and/or bottom to prevent nanotubes from passing through but allowing the solution to pass. Such nanotubes can in some embodiments be used within a chromatographic column or capillary for one or more chromatographic processes performed in a single column. Nanotubes can be contained within a container, such as a column, pipette tip, capillary, or other enclosing device. An optional retention plug or frit can in some aspects be placed at the bottom and/or top to prevent the nanotubes from being removed from the column. Such plug or frit can also consist of one or more chromatographic elements for further chromatographic purifications. These elements can include silica beads, modified silica beads, other nanomaterials, bonded silica, modified bonded silica, polymers or copolymers, modified copolymers, or other chromatographic resins or materials.

Additionally, in some embodiments, nanotubes such as those disclosed herein can be used within a chromatographic column or capillary for one or more chromatographic processes performed in a single column. The nanotubes can be within a container, such as a column, pipette tip, capillary, or other enclosing device. The nanotubes can be adhered to the walls of the enclosing container and do not prevent the flow of liquid, e.g. a sample, through the column. Rather, liquid passes through and analytes contact the nanotubes which are adhered onto the walls. Similarly, other materials can be placed at the bottom and/or top to participate in other chromatographic processes or serve other chemical or functional roles. These elements can include silica beads, modified silica beads, other nanomaterial's, bonded silica, modified bonded silica, polymers or copolymers, modified copolymers, or other chromatographic resins or materials.

A workflow or method for using the above-noted chromatographic devices or similar devices for chromatography can comprise preparing a column, conditioning the column, loading a sample, washing the sample, additional processing/washing as needed, and eluting the eluate, e.g. protein or biomolecule.

In some aspects, preparing a column can comprise assembling the desired parts as noted above and including a nanotube, e.g. TiNT, as disclosed. Conditioning the column can comprise passing a conditioning solution through the device, and/or activating, washing, hydrating, or performing other processes on the column. The conditioning of the column can in some aspects depend on the specific sample and desired chromatographic process(es). A sample can be loaded onto the column either directly by pipette or other liquid handling device or can be used inline with a syringe or can be loaded under pressure with a pump. The sample can in some aspects be allowed to interact with the medium or flow through for a given time. In samples where the desired analyte is retained by the chromatographic resin the undesired substances can be removed by washing the column with a given solution or buffer which does not result in elution of the analyte. If the column contains other chromatographic elements in series either before or after the nanotubes, the appropriate solutions can be run through the column to either concentrate, fractionate, release, or elute the substances and transfer them between phases. This can be repeated as needed until the column is ready for elution. Finally, if the sample is retained by the column it can be eluted by altering the mobile phase composition in an isochratic or gradient manner. Examples of solutions conditions which can be altered include altering chemical composition, pH, or ionic strength. Other modes of elution can also be employed such as introducing a molecule to competitively displace the analyte or introducing a chelation agent.

A chromatographic resin comprising nanotubes as disclosed herein can be employed in multiple forms. For example, such a resin can be packed within a column or microcolumn; packed inside a pipette tip through which a solution can pass through either by pipetting up or down through the resin; or packed within a container which can be attached to a syringe (such as a syringe filter) by a common syringe connection. For a syringe application, for example, the solution can be passed through either by syringe or used in-line with a pump or other device that pushes solution through the resin. In some aspects, a nanotube resin can be incorporated into a device that fits inside a centrifuge tube. A solution, e.g. a sample, can be loaded at the top of the centrifuge tube and the entire assembly centrifuged to pass the solution through the resin. Finally, a composite material comprising nanotubes can be made by polymerizing the nanotubes in a porous polymeric matrix.

By way of example and not limitation, a TiNT can be used in some embodiments for capturing, fractionating, excluding, separating, purifying, enriching, or other chromatographic separations of molecules or ions based on charge. In some aspects, a nanotube can be used to retain, exchange, or separate species based on charge polarity and charge magnitude. In some embodiments, the nanotube can either be by itself or can be used in conjugation with another resin or can be embedded within a gel matrix or other resin. The nanotube can also be covalently functionalized with other functional groups. The nanotube or variants on the nanotube listed above can in some embodiments be contained within a preparative column or other containment device and would act as the stationary phase. A sample can be introduced in an aqueous mobile phase onto the column or containment device containing the stationary phase. The desired target anions or cations can be retained on the stationary phase, depending on the pH, buffer, or other solution conditions chosen for the mobile and stationary phases. Next, elution of ions immobilized on the stationary phase can be accomplished by changing the pH conditions or by introducing additional charged species of the same charge polarity to displace the analyte ions present on the stationary phase.

By way of example and not limitation, TiNT can be used in some embodiments to separate molecules based on charge from a solution containing two or more molecules or ions with different charge magnitude or polarity. The net charge can in some aspects be varied by altering the composition of the mobile phase. Altering the pH would both change the charge on the nanotube surface and the charge on the analyte of interest and would alter the affinity of the analyte for the nanotube. The mobile phase ionic strength or choice of ion can also be modified to alter the effective interaction between the analyte and nanotube.

By way of example and not limitation, TiNT can be used for affinity chromatography of phosphopeptides or glycloproteins or other biomolecules. The nanotubes can for example be used for capture, separation, or purification of phosphopeptides or phosphorylated proteins. For example, the nanotubes can be used for the selective capture and enrichment of phosphopeptides or phosphoproteins from a cell lysate or from tryptic digested protein samples. The phosphopeptide or other biomolecule which has been immobilized can then be released either chemically by introducing a molecule with a higher affinity for the nanotubes, or by changing the solution conditions (such as pH, salt, etc). Alternatively, the titania nanotube resin containing the phosphoprotein can be directly implemented into a device which would allow the phosphoprotein to be directly analyzed with TOF-SIMS or MALDI-TOF-SIMS or another analytic technique. In either situation, the resin can likely be reused.

The disclosed TiNT, and associated binding properties for proteins and biomolecules, can in some embodiments be used for in column purification applications. For illustrative purposes, the following is a representative protocol for in-column purification and elution of phosphopeptides using the disclosed TiNT. Such a protocol or method can comprise steps such as preparing a (3-casein digest as a control, preparing a tryptic digest mixture or cell lysate, preparing a column, and conducting the purification/enrichment.

To elaborate, preparing a β-casein digest can comprise creating a solution containing: 20 uL of β-casein digest (20 ug), 100 uL of 20% acetic acid and 200 uL DI water. Preparing a tryptic digest mixture or cell lysate, can, for example, comprise adding 20 uL of each tryptic digest and then dilute with 200 uL of 20% acetic acid and 200 uL of DI water. A solution containing 20 uL of α-casein digest (20 ug), 20 uL Ribonuclease A digest (20 ug), 20 uL α-Lactalbumin digest (20 ug), 20 uL insulin digest (20 ug), 200 uL of 20% acetic acid, and 200 uL of DI water can be created. This is an example of a mixture which can be used to examine the specificity and/or selectivity of the nanotubes. To prepare a column containing nanotubes for enrichment/purification the following steps can be taken: 1) if needed, grind nanocomposite powder or nanotube alone; 2) suspend powder in water or other solvent; 3) pack into tubing or capillary either under vacuum, using a pressuring device, or manually; 4) equilibrate packed cartridge with 0.1% acetic acid; 5) load 20 ug tryptic digest of each protein prepared above; 6) wash column with 150 uL of 0.1% acetic acid; 7) wash column with 100 uL of the pH 8.3 100 mM sodium bicarbonate buffer solution solution spiked with 0.1% acetic acid and 50% acetonitrile; 8) wash column with 50 uL of 0.1% acetic acid; and 9) elute captured phosphopeptides with 100 uL of 0.5% ammonium hydroxide (pH 9.5). The flow rate for loading/washing/eluting can be about 0.5 uL/min. Using this methodology tryptic phosphopeptides of alpha- and Beta-casein can be detected on MALDI-MS. This example is intended to be illustrative of how TiNT can be used for in column purification applications, and can be readily adapted for the purification of other compounds.

In some embodiments, TiNT can be used for on target enrichment applications. The following is an example of one possible use for purification of phosphopeptides onto a patterned surface containing titania nanotubes. Such a surface can include a MALDI target (such as stainless steel) with nanotubes for phosphopeptide enrichment embedded or adhered to the surface or in contact by other mechanisms. The following is an example protocol for such an application:

(I) In-solution standard proteins digestion
1. Solutions for In-solution digestion:
   (1) Digestion buffer: Dissolve ammonium bicarbonate (50 mM final concentration, pH 7.8) in water
   (2) Reduction: Dissolve 10 mM dithiothreitol (DTT) in digestion buffer
   (3) Alkylation: Dissolve iodacetamide (40 mM final concentration) in sample solution
   (4) Trypsin stock solution: Trypsin 0.1 mg/mL in 1 mM HCl.
   Perform digestion by adding concentrated trypsin (1-2% w/w final conc) to sample solution.
2. Reduction of Proteins as standards
   (1) Dissolve a-casein, B-casein, RNaseA, BSA, and myoglobin in digestion buffer (50 mM ammonium bicarbonate+10 mM DTT)
   (2) Incubate at 56 C for 30 min.
3. Alkylation of Proteins
   (1) Dissolve iodacetamide (to a final conc. of Proteins 40 mM) into the reduced protein solution formed above.
   (2) Incubate at room temperature in dark for 1 hour.
   (3) Quench reaction with 10 mM DTT
4. Tryptic Digestion
   (4) Add concentrated trypsin to the digestion—the final Trypsin concentration should be 1-2% w/w.
   (5) Incubate at 37 C for 12 hr.
   (6) Freeze at −80 C until needed.
      (II) On-target enrichment of Phosphopeptides
1. Solutions Needed
   1. Loading Buffer: Dissolve 2,5-dihydxybenzoic acid (DHB) 50 mg/mL in 5% trifluoracetic acid (TFA) and 80% acetonitrile (MeCN)
   2. Washing Buffer: 2% TFA, 80% MeCN
   3. MALDI Matrix: Mix DHB: alpha-cyano-hydroxy-cinnamic-acid (CHCA) 4:1 (20 mg/mL: 5 mg/mL) and dissolve matrix in 50% MeCn/0.1% TFA/1% phosphoric acid
   4. Elution Buffer: 25% Ammonia Solution
2. Load Sample and Elute if needed
   (1) Dilute peptide sample (probably at least 5× v/v) in loading buffer and vortex or mix.
   (2) Deposit the sample onto the TiO2 surface and incubate at room temperature for 1 minute.
   (3) Wash the TiO2 surface with the loading solution by pipetting up and down ˜2-3 uL of the loading solution multiple times. Repeat this with the washing buffer to remove free and bound nonphosphorylated peptides.
   (4) Let the surface dry and deposit the MALDI matrix (in this case DHB:CHCA 4:1 in a 50% MeCn/0.1% TFA/1% Phosphoric acid).
   (5) Perform MALDI-MS directly on the sample.
   (6) The purified sample can also be recovered from the TiO2 by depositing an ammonium hydroxide solution and recovering the solution. This can be used for other measurement techniques such as nanoLC-ESI-MS or LC-ESI-MS/MS or other combination techniques which are sensitive to other phosphopeptides or desired analytes.
3. Regenerate Surface
   Following all uses of the surface, the TiO₂ substrate can be regenerated by immersing the substrate in ammonium hydroxide for ˜10-15 minutes. Subsequently the surface should be washed with an excess of water and ethanol and gently dried.

In some embodiments, TiNT can be used for affinity chromatography of histidine-tagged recombinant proteins. For example, in a similar design to the phosphopeptide enrichment above, nanotubes can be used to purify histidine-tagged or polyhistidine-tagged recombinant proteins. In some embodiments, an unclarified solution of histidine-tagged proteins can be passed through a nanotube-containing resin or nanotube filter, as described herein. Histidine sites can in some embodiments bind to the Ti sites on the nanotube surface and can be purified from the unclarified solution. The His-tagged proteins can then be eluted at a later time and the column reused. In some aspects, implementations of this affinity chromatography process can be used in small scale or laboratory processes but can also easily be scaled up for industrial production.

In some embodiments, TiNT can be used for affinity chromatography-metal chelate chromatography. Metal ions (such as Ti⁴⁺) can bind to proteins with exposed cysteine, histidine, and tryptophan. TiNT disclosed herein can be used to purify proteins with other amino acids exposed, in similar arrangements to those discussed hereinabove. The binding strength or affinity of the target protein to the nanotube can be altered by the choice of pH and/or buffers used. In general, elution can be accomplished by introducing a competing molecule with higher affinity for the nanotube, elution, pH change, or with imidazole or by reduction of pH.

In some embodiments, TiNT can be used for water purification applications. In some embodiments, the nanotubes as disclosed herein can be incorporated in a porous matrix or reverse osmosis filter and then used to remove contaminates from solution or undesired biomolecules.

In some embodiments, the surface of TiNT can be modified and thereafter used for fluorescent labeling. In some embodiments, the surface of TiNT can be chemically modified to introduce other functional groups such as carboxyl or amino groups using traditional conjugation techniques. Alternatively, the surface of TiNT can be modified with endiol ligands or by using a linker molecule containing a phosphonate group on one side. In some aspects, these functional groups can be used for covalent conjugation with fluorophores or coupling with other functional groups or biomolecules, such as Streptavidin. By way of example, they can be conjugated to Fluorescein isothiocyanate (FITC) or other fluorophores to allow the nanotube to be visible to a fluorescent microscope.

In some embodiments, a potential reaction for a surface modified TiNT can comprise introducing amine groups by adding a silane, such as for example Trimethoxysilylpropyldiethylentriamine, to nanotubes, followed by acidification to pH 4.0 and heating at 75° C. for 3 hours. Other silanes are suitable here as well since organosilanes will form RnSiX(4-n) where X is a hydrolyzable group (alkoxy, acyloxy, amine, chlorine, methoxy, ethoxy) R is a non hydrolyzable organic radical. By way of example and not limitation, suitable silanes can comprise N-(2-Aminoethyl)-3-aminopropylmethyldimethoxysilane, N-(2-Aminoethyl)-3-aminopropyltrimethoxysilane, 3-Aminopropylmethyldiethoxysilane, 3-Aminopropyltriethoxysilane, 3-Aminopropyltrimethoxysilane, (N-Trimethoxysilylpropyl)polyethyleneimine, and Trimethoxysilylpropyldiethylenetriamine. The nanotubes can then be washed with water until a pH of about 7 is reached. The amine terminated nanotubes can then be dried.

In some embodiments, a potential reaction for a surface modified TiNT can comprise introducing carboxyl groups to the dry product of the above reaction. The dry amine-modified nanotubes can be mixed with dry dimethylformamide (DMF) to wash them. The nanotubes sediment and the DMF can be removed by pipette. Following this, dried DMF is added and nitrogen has can be bubbled through the solution. Subsequently, succinic anhydride can be added and the solution can be stirred under nitrogen for about 8 hours. The DMF can then be removed and the nanotubes can be washed in water until a pH of about 7 is reached. The carboxyl modified nanotubes can then be stored at 4° C. These nanotubes can then be lyophilized or dried under vacuum for future use.

In some embodiments, Streptadivin or other biomolecules can be conjugated to the product of the above reaction. Dry product from the above reaction can be thoroughly washed with working buffer, such as for example MES, and pelleted by centrifugation. Subsequently, the pellet can be resuspended in a fresh buffer containing N-hydroxysuccinimide or N-Hydroxysulfosuccimide and 1-Ethyl-3[3-dimethylaminopropyl]carbodiimide hydrochloride (EDAC) or another suitable water-soluble carbodiimide. After mixing for about 1 hour at room temperature, the supernatant can be removed and the nanotubes washed with water. The pellet can then be resuspended in PBS or another suitable buffer. Streptadivin can then be added to the sample to couple.

In some embodiments, TiNT can be used as a nucleating agent or nucleation site for protein crystallization. In some embodiments, TiNT as disclosed herein can be used as an immobilization site or as a nucleant to promote protein crystallization. In some embodiments, multiwell microplates containing nanotubes in solution or nanotubes which were bound to a substrate inside the microplate can be used in screening studies for protein crystallization.

In some embodiments, the nanotubes can also be used to help crystallize or immobilize large amounts of protein for other types of measurements. For example, the nanotubes can be used as a substrate to immobilize large amounts of protein for electron microscopy or other imagining or analytical measurement techniques. Alternatively, in some embodiments nanotubes as disclosed herein can be used in processing biomolecules, such as in lyophilization.

In some embodiments, the TiNT as disclosed herein can be used to transfect or assist in transfection of cells. Indeed, the disclosed TiNT is not cytotoxic to a number of cell lines, thereby making them suitable for such uses.

The disclosed nanotubes can be used as a device for delivering or recovering large amounts of biomolecules to or from a cell. For example, modifying or assembly modifying the nanotubes such that they are pH sensitive can allow for the nanotubes to be triggered to release at a given intracellular pH. Nanotubes can then be used to deliver biomolecules in transfection reactions.

In some embodiments TiNT as disclosed herein can be used to stabilize therapeutic biomolecules for transport or delivery. Biomolecules immobilized on the nanotubes can have increased enzymatic lifetimes and/or decreased degradation rate. Utilizing the nanotube in given formulations can enhance the shelf-life of biologics or other medicines.

In some embodiment, TiNT surface hydration properties can be modified by sonication, mechanical processing, or the addition of defect sites. Careful control of this process can be used to create a variety of substrates which have different interactions with proteins.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Materials and Methods for Examples 1-11

### Chemicals

Chromatographically purified lyophilized Bovine Pancreatic Ribonuclease A (RNaseA), lyophilized hen's egg white lysozyme, titanium(IV) n-butoxide (TNBT; 97%) and Hydrofluoric Acid (HF, 48 wt % in H₂O) were purchased from Sigma Aldrich (St. Louis, Mo., United States of America). 1 NI HEPES buffer (pH 7.2) and NaOH were purchased from Fisher Scientific (Hampton, N.H., United States of America). 32 nm anatase TiO₂ nanoparticles were purchased from Alfa Aesar (Ward Hill, Mass., United States of America). All chemicals were used without further purification.

Nanomaterial Synthesis

Titania nanotubes were hydrothermally synthesized and shortened as previously described (Mogilevsky et al., 2008(b); Chen et al., 2009). Briefly, 32 nm anatase nanoparticles (4 g) were added to freshly prepared 10 M NaOH (400 mL). The mixture was then sealed in a PTFE-lined stainless steel autoclave and maintained at ˜135° C. for 72 hr. The resulting material was repeatedly washed with distilled water and HCl (0.1 M) until the supernatant reached a pH of 5-6. Subsequently, the nanotubes were shortened by wet ball milling in a laboratory ball mill (Glen-Mills, Clifton, N.J., United States of America). Approximately 65 mL of the aforementioned nanotube suspension was mixed with 30 g of 100 μm diameter ZrO₂ beads (Glen-Mills) in a grinding vessel and ground for 45 minutes. The grinding vessel was surrounded by a cooling bath which kept the grinding vessel temperature below 100° C. Following ball milling, the supernatant, which contains only shortened nanotubes, was decanted and centrifuged to remove any excess grinding media. The resulting suspension was filtered through a 0.2 μm PES membrane filter (Millipore, Billerica, Mass., United States of America). The nanotube concentration was determined by thermogravimetric analysis on a Q5000IR TGA (TA Instruments, New Castle, Del., United States of America).

Anatase nanotiles were synthesized similarly to previous publications (Han et al., 2009). Titanium(IV) n-butoxide (21 mL, 0.579 mol) and Hydrofluoric Acid (1.6 mL, 0.005 mol) were combined in a PTFE-lined stainless steel autoclave and maintained at ˜180° C. for 24 hours. The resulting precipitate was repeatedly washed with ethanol and distilled water. Subsequently it was dried under vacuum and calcinated in air at 400° C. for 1 hour. The resulting precipitate was dispersed in water and dialyzed against a 500-fold excess of 25 mm HEPES for 72 hours; the dialysate was exchanged every 24 hours.

Quantitative Adsorption Measurements

Protein solutions were prepared immediately prior to use by dissolving a known weight of protein in a given volume of 25 mM HEPES (pH 7.2). The solution was filtered through a 100 nm PES membrane filter to remove any preexisting aggregates. Samples were prepared by combining a fixed amount of nanotube with a varying amount of protein and buffer in low protein-binding centrifuge tubes; the total sample volume was held constant. Samples were mixed on a rotisserie rack for 7 days at room temperature. A depletion method was used to determine the amount of protein bound to the nanotubes. The nanotubes and protein-nanotube conjugates were first pelleted by centrifugation, leaving the unbound protein in the supernatant. The protein concentration in the supernatant was assayed with the Quant-IT Protein Assay (Invitrogen, Carlsbad, Calif., United States of America). All measurements were performed in triplicate at 23.6° C. on a SpectraMax384 Microplate Reader (Molecular Devices, Sunnyvale, Calif., United States of America) in a black 96 well microplate (Brandtech, Essex, Conn., United States of America), and analyzed using SoftMax Pro (Molecular Devices) following the manufacturer's instructions.

Enzymatic Activity Assay

Samples of RNaseA-TiNT conjugates at different RNaseA/TiNT molar ratios were prepared similarly to the adsorption experiments, except in these experiments, the concentration of RNaseA was fixed and the TiNT concentration varied. Serial dilutions of the samples were prepared and assayed with a fluorescence assay (RNaseAlert, Integrated DNA Technologies Inc., Coralville, Id., United States of America) per the manufactures directions. Measurements were performed in triplicate at 37° C. in a Spectramax384 microplate reader (Molecular Devices). Measurements were taken every 60 seconds.

SDS-PAGE (Denaturing Gel Electrophoresis)

SDS-PAGE measurements were performed on a NuPAGE 8-16% Bis-Tris Gradient Mini Gel (Invitrogen) with an MES running buffer. Samples were pelleted by centrifuge, resuspended in 1× NuPage LDS Sample Buffer, and subsequently denatured at 70° C. for 10 minutes. The volume of sample loaded into the gel varied between 15-30 μL, depending on the trial and anticipated concentration. A seven-protein molecular weight marker (GE High-Range Molecular Weight Marker, GE Healthcare Life Sciences, Piscataway, New Jersey, United States of America) was run in one lane on each gel to calibrate the molecular weight migration pattern. Gels were run on an XCell SureLock Mini-Cell (Invitrogen) at 200 V for 35 minutes. Following electrophoresis, gels were stained with colloidal Coomassie G-250 (SimplyBlue Safestain, Invitrogen) per the manufacturer's directions for high-sensitivity staining. The gels were subsequently scanned at 600 dpi using a desktop flatbed digital scanner (HP ScanJet, Hewlett-Packard, Palo Alto, Calif., United States of America) and dried between cellulose film for storage (Pierce Gel Drying Kit, Thermo Scientific, Rockford, Ill., United States of America). The scanned images were cropped and the brightness and contrast were uniformly adjusted to increase clarity for the reader. No other image adjustments were performed.

Electron Microscopy

To prepare TEM samples, the reaction mixture was drop deposited onto a 300 Mesh lacey carbon grid (Ted Pella, Redding, Calif., United States of America) and allowed to dry in air. High-resolution TEM imaging was performed at 200 kV on a JEOL 2010E-FasTEM. Images were acquired using a 2 k×2 k Gatan CCD bottom mount camera. When indicated, pre-deposited samples were stained for 3 minutes in an aqueous solution of Uranyl Acetate (2% w/v). The grid was subsequently washed to remove excess Uranyl Acetate and reimaged. Samples for SEM were drop deposited onto a clean Si square (Ted Pella). SEM imaging was performed on a Hitachi S-4700 field emission SEM at 5 kV and a FEI 600 Helios NanoLab DualBeam at 15 kV. Prior to imaging at 15 kV, a 2.5 nm thick layer of Au/Pd was sputtered onto the sample in an Argon plasma (Cressington Scientific Instruments Ltd, Watford, England).

Dynamic Light Scattering and Zeta Potential Measurements

Dynamic light scattering (DLS) measurements were performed in a backscattering geometry on a Malvern Zetasizer Nano ZS (Malvern Instruments Ltd., Worcestershire, United Kingdom) using either a disposable folded capillary cell (Malvern) or a disposable low-volume sizing cuvette (Brandtech). Measurements were performed in triplicate at 25.0° C. and allowed to equilibrate for a minimum of 120 seconds prior to measurement. Measurements were analyzed in software provided with the instrument using cumulant analysis. Zeta potential measurements were performed in triplicate using the disposable folded capillary cell (Malvern) on a Malvern Zetasizer Nano ZS equipped with a MPT-2 Autotitrator (Malvern), degasser, and liquid filled glass combination electrode. Automated pH titration was employed for isoelectric point measurements. Standardized 1N HCl and 1N NaOH solutions (Sigma Aldrich) were used to create 10 mM and 50 mM solutions of HCl and NaOH, which were used as titrants.

Thermogravimetric Analysis

Anatase nanomaterials were dried in a vacuum oven (Yamato ADP-21, Yamato Scientific America Inc., Santa Clara, Calif., United States of America) at 50° C. overnight and equilibrated under ambient conditions (RH ˜40%) for a minimum of 30 days. TGA was performed with a Discovery TGA (TA Instruments, New Castle, Del., United States of America) in platinum pans. Measurements were performed at 10° C.min⁻¹ from 45° C. to 600° C. using a dry nitrogen atmosphere with a constant flow rate of 10 mLmin⁻¹.

### Example 1

### Adsorption of Ribonuclease A on Titania Nanotubes

Shortened titania nanotubes (TiNT), illustrated in FIG. 1, were produced by a hydrothermal synthesis, as previously described (Mogilevsky et al., 2008(a); Mogilevsky et al., 2008(b). The TiNT were then dispersed in 25 mM HEPES buffer (pH 7.2) at a final concentration of 75 μgmL⁻¹. The resulting nanotubes typically consist of 4 layers of anatase (001) rolled around the anatase [010] axis and have a typical outer diameter and length of 12 nm and 100 nm, respectively (Mogilevsky et al., 2008(a); Mogilevsky et al., 2008(b). The nanotubes can have an isoelectric point of pH 2.7 and form a stable dispersion at physiological pH. Chromatographically purified Bovine Pancreatic Ribonuclease A (RNaseA) was prepared by dissolving lyophilized protein in 25 mm HEPES buffer (pH 7.2), at a typical concentration of about 2 mgmL⁻¹. The protein solutions and nanotube dispersion were then filtered through 100 nm and 200 nm membrane filters, respectively, to remove any preexisting aggregates. Protein and nanotube aggregates were not detected with dynamic light scattering, nor were any protein oligomers detected by gel electrophoresis (SDS-PAGE).

### Example 2

### Characterization of the Interaction Between RNaseA and TiNT

The interaction between RNaseA and TiNT was characterized by performing quantitative adsorption measurements at room temperature. Here, the concentration of RNaseA was varied while holding the nanotube concentration and the total volume constant, as illustrated in the inset of FIG. 4. The samples were gently mixed for 7 days on a rotisserie rack to ensure they had reached equilibrium. Subsequently, the protein-nanotube conjugates were pelleted in a laboratory centrifuge and the amount of protein remaining in the supernatant was measured using a fluorescent assay. Low protein binding centrifuge tubes were used in all steps of the experiment to minimize non-specific protein adsorption on the sample walls. In protein-only controls, the amount of protein loss due to adsorption on the sample container walls or during centrifugation was not statistically significant.

RNaseA is a highly stable globular protein and is not expected to denature, aggregate, or oligomerize under the disclosed experimental conditions. The exposed surface area of TiNT is 119.3 m² g⁻¹ and was used to calculate the adsorption isotherm of RNaseA per unit surface area of TiNT. As shown in FIG. 4, the adsorption capacity reaches as high as 563.3±0.9 (pmol RNaseA)(m² TiNT)⁻¹. This is over 1,000 times the expected value of a close-packed RNaseA monolayer on the nanotube surface, which is 0.48 (μmol RNaseA)(m² TiNT)⁻¹ (see Equation 3). The observed capacity cannot be accounted for by confinement of the protein in the nanotube's interlayers, which are 8.0 Å wide, or inside the nanotube, which can only fit a single row of RNaseA.

To elaborate, the geometric surface coverage of RNaseA on the nanotube was estimated by modeling the protein as a hard sphere of diameter a, and the nanotube with an outer radius of r₀ and length l. This model neglects conformational change of the protein and steric effects, thus allowing for a greater number of proteins on the surface than would be physically realized. The ellipsoidal shape of the protein was also neglected, assuming it to be a slightly larger sphere with a length equivalent to the largest diameter of RNaseA. These assumptions over predict the number of proteins predicted to reside per layer, as the minor difference in size does not adequately compensate for the significant steric effects that experimentally limit the monolayer capacity. Therefore this model predicts fewer layers than actually would be realized, but provides a reasonable model to calculate the approximate number of layers.

To compute the number of protein in n layers, the surface area of each exposed layer available for protein binding was calculated and the projected area occupied by the spherical protein was used to determine the binding capacity. Assuming each layer to be densely packed, a nanotube of length/and radius r₀ has an external surface area of 2πr₀l. Following the adsorption of a single protein layer the new surface area available for binding will be 2π(r₀+a)l, where a is the diameter of the protein.

Extending this for n layers:

$$Total Surface Area = \begin{matrix}\begin{matrix}n \\ \end{matrix} \\ i = 1\end{matrix} 2 π ( {r}_{0} + ( i - 1 ) a ) l = nl π ( 2 {r}_{0} + ( n - 1 ) a ) \qquad ( Equation 1 )$$

The length dependence of this equation can be removed by normalizing Equation 1 by the nanotube. The simplest unit, which the delaminated anatase nanotube structure can be constructed from, is from four concentric layers of TiO₂ contains 954 TiO₂ molecules. This unit has a mass of about 1.25×10⁻¹⁹ g, a length of about l=0.380 nm, and a radius of about r₀=6.25 nm. Therefore the surface area per gram nanotube in units of m²/g is:

$$\frac{S . A .}{g nanotube} = 9.55 \frac{{m}^{2}}{g {nm}^{2}} ( 12.5 nm + ( n - 1 ) a ) a \qquad ( Equation 2 )$$

Defining the protein surface density σ(units of protein/m²) and converting to umol/g, provides:

$$\frac{μ mol Protein}{g nanotube} = ( 1.59 \times {10}^{- 17} \frac{{m}^{2} μ mol}{g {nm}^{2} ( molecules protein )} ) σ ( 12.5 nm + ( n - 1 ) a ) a \qquad ( Equation 3 )$$

Assuming the diameter of RNaseA is 4.4 nm, the projected surface area is:

$${π ( \frac{4.4 \times {10}^{- 9} m}{2} )}^{2} = 1.52 \times {10}^{- 17} {m}^{2} \qquad ( Equation 4 )$$

corresponding to a surface density of σ=(molecule RNaseA)/(1.52 10⁻¹⁷ m²).

Based on this the monolayer coverage (n=1) corresponds to 57.5 μmol RNaseA/nanotube. Noting the external surface area of the nanotube (about 119.3 m²/g) monolayer coverage corresponds to 0.48 (μmol RNaseA)/(m² nanotube).

Further inspection of FIG. 4 reveals that adsorption occurs in two distinct regimes which depend on whether the equilibrium concentration of the unbound protein, c, is less than or equal to a critical concentration, c*=23 μM. For c<c*, the isotherm exhibits a step like or sigmoidal behavior, quickly reaching a plateau at about c=2.5 μM with a corresponding surface coverage of ˜10 (μmol RNaseA)(m² TiNT)⁻¹, equivalent to ˜55 concentric layers of protein. The isotherm remains flat until c>13 μM. At c*, the unbound protein concentration stops increasing and any additional protein is adsorbed. As more protein is added above c*, the surface coverage rapidly increases from ˜190 μmol RNaseA/m² TiNT to as high as 563.3±0.9 (μmol RNaseA)(m² TiNT)⁻¹. The rapid increase of adsorbed protein, observed when the unbound protein concentration is increased above c*, is typical of systems undergoing self assembly or macromolecular condensation.

### Example 3

### Self Assembly of RNaseA-TiNT

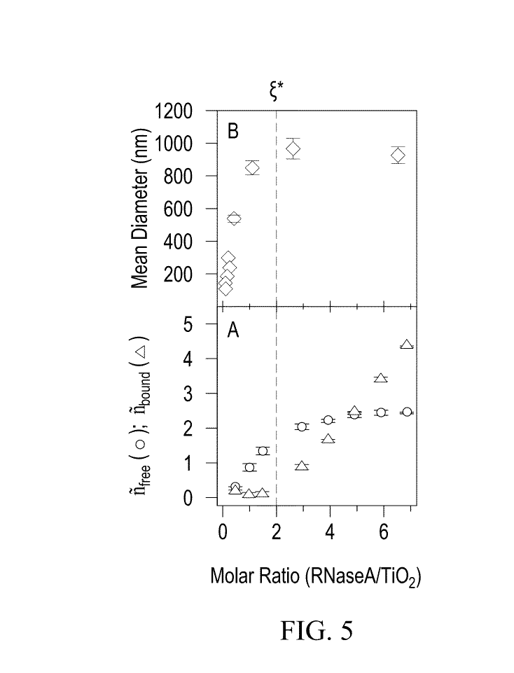

Self assembly is a structural phase transition that occurs when, upon reaching a critical concentration, it becomes more energetically favorable for any additional solute to aggregate rather than remain free in solution. In FIG. 5A, the possibility of this scenario is investigated by plotting both the normalized concentrations of adsorbed and unbound RNaseA versus ξ, which is the ratio of moles of RNaseA to moles of TiO₂ contained in the sample. FIG. 5A is the adsorption isotherm showing the relative number of nanotube-bound (Δ) and unbound (free) (o) protein. As seen in FIG. 5A, at low values of ξ the amount of unbound RNaseA increases steadily while the amount of adsorbed RNaseA remains at a relatively low fixed value. This behavior continues until ξ reaches a critical aggregation concentration (CAC), ξ*, at ξ*=2. After reaching the CAC, the amount of adsorbed RNaseA increases linearly while the amount of unbound RNaseA reaches a steady value. This adsorption behavior and the clear CAC are both consistent with the system undergoing self assembly starting at around ξ*.

The structural changes associated with self assembly should result in observable size increase near ξ*. Dynamic light scattering (DLS) was used to investigate how the size of the RNaseA-TiNT conjugates scales with ξ. FIG. 5B includes the DLS measurement showing that the mean aggregate diameter increases with ξ. The dashed line is drawn to indicate the critical aggregation concentration, ξ*. These measurements provide an effective mean hydrodynamic diameter for each sample which is characteristic of the size distribution of the resulting aggregates. Independently, RNaseA and the TiNT have mean hydrodynamic sizes of 4.8 nm and 113 nm, respectively. However, as seen in FIG. 5B, the addition of protein results in the formation of RNaseA-TiNT conjugates which are both larger than the individual component and whose size is strongly dependent on ξ.

In agreement with the adsorption measurements, DLS reveals a structural transition around ξ=2 as well as two distinct growth regimes. Below ξ=0.05, the hydrodynamic size remains nearly constant at ˜125 nm, while above ξ=0.05, the size grows exponentially until saturating near ξ=2 at ˜1000 nm. In conjunction with the multilayer loading capacities indicated by our adsorption measurements, these findings suggest that the size increase may be due to the formation of much larger structures containing multiple nanotubes, forming either through interaggregate interactions or self assembly.

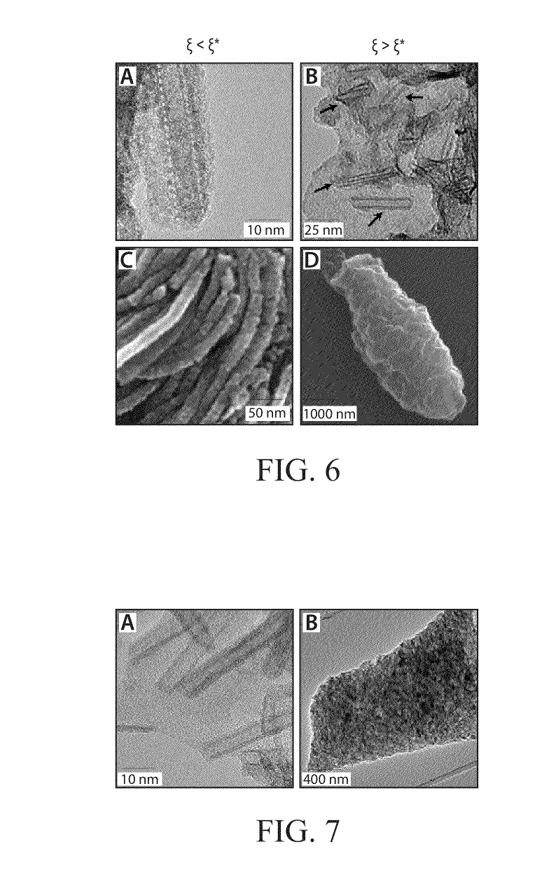

TEM and SEM imaging of the RNaseA-TiNT conjugates further support a scenario of multilayer adsorption at low ξ, followed by the formation of larger structures at higher ξ. As shown in FIGS. 6A and 6C, TEM and SEM images of the protein-nanotube conjugates formed at ξ=0.06 clearly show a 6-8 nm thick adsorbed layer of RNaseA on the nanotube surface. This thickness is nearly twice the diameter of the protein and is consistent with the formation of protein multilayers on the nanotube. FIGS. 6B and 6D show TEM and SEM images of aggregates formed above the CAC, at ξ˜2.1. The TEM (FIG. 6B) image reveals the presence of a large aggregate cluster consisting of multiple nanotubes embedded in a large protein plaque, while the SEM image (FIG. 6D) reveals a prolate ellipsoidal aggregate that is 2 μm wide and 6 μm long. These images also show that that protein multilayers form around the entire TiNT, including the open ends, where there is no accessible nanotube surface for adsorption. Here the proteins must adsorb by associating with adjacent, previously adsorbed protein, and would require a driving force to overcome the significant interprotein Coulomb repulsion.

### Example 4

### Self Assembly of Lysozyme-TiNT

Similar micron-sized, self-assembled aggregates were also observed with Lysozyme, which has a size and shape similar to RNaseA, but is more highly charged at physiological pH. High resolution TEM imaging, shown in FIG. 7A, reveals multiple nanotubes embedded in a thick lysozyme plaque, while FIG. 7B shows the formation of micron-sized aggregates of TiNT and lysozyme similar to those found in our RNaseA-TiNT experiments. These findings suggest that the nanotube may act as template for the self-assembly of larger protein-based materials.

### Example 5

### Enzymatic Activity of RNaseA-TiNT Assemblies

Results provided herein demonstrate that that this process can be exploited to create functional, insoluble enzyme biocatalysts. The enzymatic activity of the multilayer and self-assembled RNaseA-nanotube conjugates was assessed with a quantitative assay (RnaseAlert, IDT Inc.). The assay consists of an oligonucleotide substrate which has a fluorescent reporter and dark quencher attached at opposite ends. Enzymatically active RNaseA catalyzes the cleavage of the phosphodiester bond between the 3′-PO₄ end of pyridine and the 5′-OH of the adjacent nucleotide,³² separating the fluorescent reporter from the quencher, and restoring fluorescence. A series of samples containing identical RNaseA concentrations and different TiNT concentrations was incubated with an identical and excess amount of the oligonucleotide substrate.

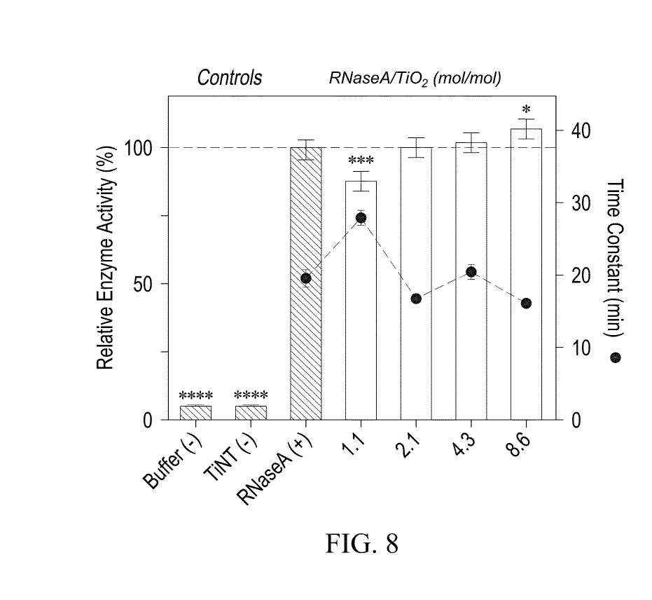

In FIG. 8, the fluorescent intensity, which is directly proportional to the amount of cleaved substrate, was compared after incubating the samples and substrate for 1 hour at 37° C. At ξ=1.1, RNaseA is adsorbed as multilayers on TiNT and here the enzymatic activity is 88.7% of the protein control. This reduction, typical for carrier-bound enzymes, can be due to incorrect orientation of the active site, His-119, or structural modifications resulting from immobilization.

In contrast, the activity of self-assembled samples was greater than or equal to that of the native enzyme's activity. For instance at ξ=8.6, an enhanced activity of 107.4% (p<0.05) was observed. The differing activity of the multilayer and self-assembled states suggests that the orientation or packing of RNaseA in these two states may also differ. In adsorption measurements (FIG. 5A) it was found that the amount of protein bound scaled with ξ. Similarly, the enzymatic activity also appears to increase with ξ, suggesting self assembly may act to increase the active lifetime of adsorbed enzymes or increase the accessibility of adsorbed RNaseA by forming a more ordered or less tortuous assembly. The retention of activity suggests that this technique can be useful for assembling functional biocatalytic materials.

### Example 6

### Effective Diffusivity and Microstructure of RNaseA-TiNT Assemblies

The enzyme's activity occurs over a characteristic timescale, τ, determined by the effective diffusivity, Dₑ, of the oligonucleotide through the immobilized protein layers. Dₑ is sensitive to the microstructure of the immobilized layer and will be decreased in layers with a lower porosity, φₚ, or an increased tortuosity, T. Therefore, measurements of τ, which is dependent on Dₑ, serve to probe the microstructure of the immobilized layer, and will scale as τ∝T/φₚ.

The enzymatic reaction exhibits first order kinetics; τ is obtained by fitting the time dependence of the fluorescent intensity for the first 60 minutes. As shown in FIG. 8, τ for the multilayer adsorbed protein (ξ=1.1) is significantly larger than the protein-only control (p<0.001). In contrast, τ for the self-assembled sample occurring at ξ=8.6 was slightly lower (p<0.05) than the protein-only control. The other self-assembled samples did not have a statistically significant difference from the protein-only control. Interestingly, τ and relative enzymatic activity appears to be correlated. Compared to the protein-only control, the relative activity was lower when τ was increased, enhanced when τ was decreased, and unchanged when τ was the same as the control. The kinetic differences suggest enzyme immobilized in the multilayer and self-assembled states have a different microstructure.

The relative activity is a function of the active lifetime of the enzyme and accessibility, while the reaction kinetics are an indicator of the diffusional resistance the substrate experiences. A more porous, or less tortuous immobilized layer would increase the enzyme accessibility and substrate diffusivity, resulting in enhanced activity and τ similar to the bulk protein. These measurements suggest that self assembly alters the microstructure of the immobilized multilayers, forming either a more porous or less tortuous network of immobilized proteins than is found in the multilayer state. As ξ increases, more protein is immobilized and the number of protein residing on the exterior of the self-assembled aggregates also increases. As the aggregate surface area increases, reactions more frequently occur on the surface and more collisions between substrate and the enzyme occur, shifting the reaction kinetics from diffusion limited to reactant limited regime.

### Example 7

### Gel Electrophoresis of Adsorbed Protein

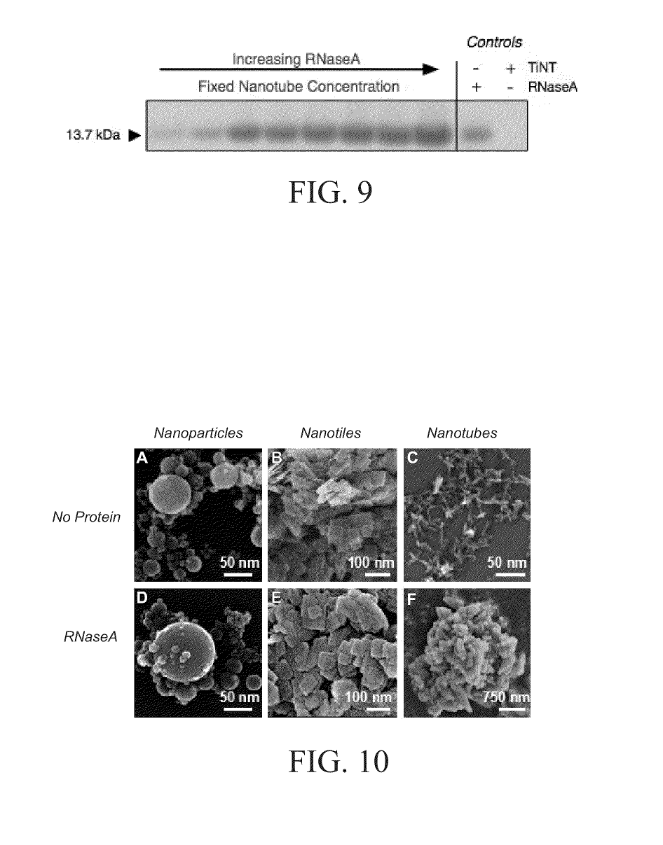

Protein adsorption on nanoparticles has been shown to cause protein fibrillation and anomalous aggregation. Therefore, denaturing gel electrophoresis (SDS-PAGE) was performed on the pelleted protein-nanotube conjugate to investigate whether protein oligomers were formed in the process of assembly. As shown in FIG. 9, only a single band, corresponding to the RNaseA monomer mass of 13.7 kDa, is visible in the sample lanes, demonstrating that the bound protein does not oligomerize. The bands are shown in order of increasing ξ, for a fixed nanotube concentration; the band intensity corresponds to the amount of protein bound. The intensity pattern shows that the amount of protein bound increases with ξ and agrees qualitatively with our DLS and adsorption measurements. Extensive washing of the pellet did not affect the band pattern or intensity, indicating that the protein is strongly bound to the nanotube and confirming that the bands observed do not correspond to residual unbound protein. SDS-PAGE of the supernatant also only contained monomers, indicating that the nanotube does not act as a nucleant for oligomerization of the unbound protein. SDS is only able to denature protein structures formed by non-covalent bonds and was able to easily solubilize the nanotube-bound protein in both the multilayer and self-assembled aggregate states, suggesting that the immobilization is non-covalent.

### Example 8

### Role of TiNT Surface Chemistry

Immobilized transition metals can interact with amino acids, and non-covalent interactions between transition metal ions and protein surface residues can modify protein-protein interfacial interactions. As seen in FIGS. 2A-2C, the exposed surface of TiNT is anatase (001)-like, formed by delaminating anatase along the [001] direction and curving the delaminated anatase (001) surface around the [0 1 0] axis. The surface Ti sites on clean bulk (001) surface are all fivefold coordinated and under ambient conditions these sites are hydroxylated by dissociative water adsorption.⁴⁴ In contrast, water is only molecularly adsorbed on the surface of the nanotube, which also contains only fivefold coordinated Ti sites. The stability of these groups against hydroxylation leaves these groups open to react and is crucial its reactivity.

Therefore, to understand if the unique surface chemistry of TiNT can contribute to the phenomena observed, similar experiments were run to examine the interaction between RNaseA with additional anatase nanomaterials, particularly anatase nanotiles (which contain a hydroxylated anatase (001) surface) and commercial anatase nanoparticles (which primarily have an anatase (101) surface), as depicted in FIGS. 2A-2C. As seen in FIGS. 10A-10F, with the exception of the TiNT, the interaction between RNaseA and other anatase nanomaterials did not appreciably change the size or dispersion of the nanomaterials. The assembly of larger aggregates formation only occurred with the nanotubes (FIGS. 10C and 10F). This suggests that the unique surface chemistry of the nanotube can be crucial to the production of these functional protein-based materials.

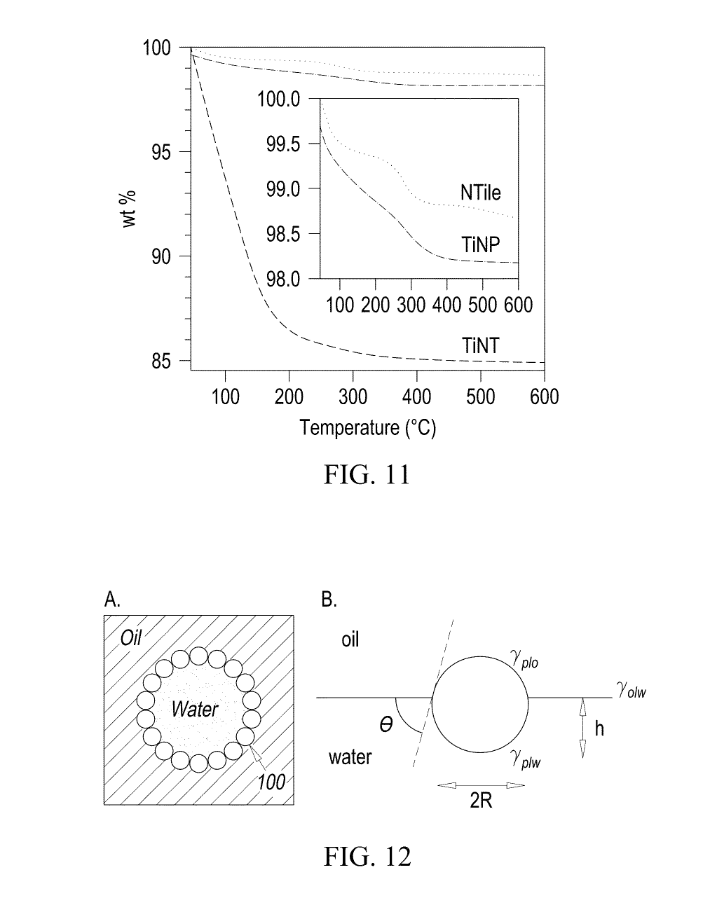

Thermogravimetric analysis (TGA) of the dried nanomaterials, following their exposure to ambient conditions, clearly highlights their different hydration properties. As seen in FIG. 11, the nanotubes exhibit only a single weight loss between 50° C. to 150° C., corresponding to the loss of molecularly adsorbed water. On the other hand the nanotiles have two different distinct weight losses, one occurring between 50° C. and 100° C., corresponding to the evaporation of bulk water, and an additional weight loss near 275° C., due to the removal of the surface hydroxyl groups. In contrast, the nanoparticle weight linearly decreases between 50° C. and 300° C. Anatase nanoparticles typically contain a large number of defect sites on the low-energy (101) surface and frequently expose a variety of additional crystal facets. The continuous loss is consistent with the large number of energetic environments on the nanoparticle surface and the removal of molecularly adsorbed water with different hydrogen bonding configurations.

Although both the nanotubes and nanotiles expose the anatase (001) surface, aggregation was only observed on the nanotube. This suggests that this phenomenon requires more than just the presence of the anatase (001) surface. The difference between these two materials lies in the stability of the surface Ti groups against hydroxylation—while both expose the anatase (001) surface, only the nanotube contains exposed and stable undercoordinated Ti surface sites. The bond strain induced by the nanotube's curvature can be essential for preventing hydroxylation of the nanotube surface. When this is removed the undercoordinated Ti groups are instantly hydroxylated and no longer available to react. These findings suggest that the exposed, stable, undercoordinated Ti sites on the nanotube surface is crucial to initiating the self association of the free and bound protein.

### Example 9

### Free Energy Change Associated with Self-Assembly

Without being bound by any particular theory or mechanism of action, the results disclosed herein suggest a model for the observed RNaseA-TiNT interactions that is illustrated in FIGS. 3A-3D. Initially, the system consists of monomeric protein and individual nanotubes coated with RNaseA. At extremely low protein concentrations (ξ<<ξ*), protein should adsorb as monolayers (FIG. 3A). As the protein concentration is increased, extensive protein multilayers form on the nanotubes (FIGS. 3B and 3C), continuing until a critical concentration of free protein is reached. Above the critical concentration excess protein and the dispersed individual protein-nanotube conjugates assemble into an aggregate mesophase consisting of large, prolate ellipsoidal structures that contain multiple nanotubes and proteins (FIG. 3D).

This adsorption behavior, as shown in the disclosed adsorption measurements (FIG. 5A), is a hallmark signature of self assembly. Similar phenomenon involving the emergence of an aggregate mesophase is observed in the formation of supramolecular assemblies and in other self-assembling systems such as liposomes or giant vesicles. Thermodynamically, the structural transition between the dispersed (FIG. 3C) and self-assembled state (FIG. 3D) is favorable only if assembly reduces the Gibb's free energy of the system. The critical aggregation concentration (CAC) at which this transition occurs is determined by the chemical potential difference of any two phases in the system. This allows the CAC to be written in terms of the chemical potential of the protein monomer, μ₁, and the aggregates, μ_{N},

$$C A C \approx exp ( \frac{- ( {μ}_{1} - {μ}_{N} )}{{k}_{B} T} ) . \qquad ( Equation 5 )$$

The chemical potential difference, μ₁-μ_{N}, is the Gibb's free energy change associated with the emergence of the aggregate mesophase, AG. From the data disclosed in FIG. 5 it was estimated that the critical molar ratio at which the mesophase emerges is ξ*=2.0. At mesophase emergence, there are 0.35 mole RNaseA adsorbed per mole of TiO₂, corresponding to a surface coverage of 36.7 (μmol RNaseA)/(m² TiNT), nearly 100 times that of monolayer surface coverage. In the disclosed experiments the CAC corresponds to a RNaseA monomer concentration of 15.3 μM. From this, it was determined that the emergence of the mesophase results in a reduction in free energy of approximately:

ΔG=−k_{B}T ln(CAC)≈15.1k_{B}T (Equation 6)

At the critical transition, the packing limits for the protein on the nanotube surface have been reached. This forces the system to rearrange and reassemble through interaggregate interactions which reduce the Gibbs free energy by 15.1 k_{B}T. The inhomogeneous microstructure and prolate shape of the observed aggregates are characteristic of binary supraself-assembled systems. The interpenetrating packing of the multilayer-coated nanotubes observed with TEM (FIG. 6B) allows for a higher packing volume fraction, while the prolate shape decreases the Gaussian curvature and reduces the interfacial tension.

### Example 10

### Adsorption of Proteins

Adsorption experiments were performed with RNaseA as well as two additional globular proteins, Hen's Egg White Lysozyme (Lysozyme) and Human Ubiquitin (Ubq). As detailed in Table 1, Lysozyme and RNaseA both have comparable sizes and masses, while Ubiquitin is about 35% lighter.

TABLE 1
Various physical properties of RNaseA, Lysozyme and Ubiquitin

| Property | Ribonuclease A | Lysozyme | Ubiquitin |
|---|---|---|---|
| Molar Mass (Da) | 13686.63 | 14313.14 | 8564.84 |
| Number of Residues | 124 | 129 | 76 |
| Specific Volume at 25° C. | .704 | .702 | .743 |
| Dimensions (Å³) | 38 × 28 × 22 | 45 × 30 × 30 | 51 × 43 × 29 |
| Isoelectric Point (pH) | 9.4 | 11 | 6.79 |
| Charge at pH 7.2 (+e) | 6.29 | 8.97 | 0.96 |
| Isothermal Compressibility | .112 | .467 | ??? |
| (m² N⁻¹) | | | |
| Structure - % α-helix | 11.5 | 29 | 16 |
| Structure - % β-sheet | 33 | 6 | 37 |
| Monolayer Coverage | 0.48 | 0.47 | 0.68 |
| (theory) (μmol m⁻²) | | | |

A protein's isoelectric point (pI) is defined by the pH at which it has no net charge; above the pI the protein will carry a positive charge, below it will carry a negative charge. The amino acid (AA) composition of the protein and the acid-base properties (pKa) of the AA groups are well known. From this one can reasonably approximate the net charge on the proteins as a function of pH. Experimentally, Lysozyme and Ubiquitin both have significantly different isoelectric points than RNaseA. Ubiquitin has an experimental isoelectric point of pH 6.79 and will have a slightly negative surface charge at pH 7.2. Lysozyme's isoelectric point is significantly higher, occurring at pH 11, and will have a larger positive surface charge than RNaseA at pH 7.2.

This allows for the examination of the role of charge in adsorption. From the amino acid composition and known pKa of the amino acid groups in each protein, the net charge was calculated as a function of pH, utilizing software which automated the calculation when provided with the AA sequence obtained from a protein data bank.

The net charge on all three proteins was plotted as a function of pH. It was evident that at pH 7.2, Lysozyme had a more significant positive charge (+8.97e) than RNaseA (+6.29e), while Ubiquitin had a near-unity charge (+0.96e). It should be noted that these differ slightly from the experimentally observed properties. It does not account for which residues are exposed or for post-translational modifications that would also modify the charge. These estimates provide an approximation of the charge. This is evident from the calculated properties of Ubiquitin, which experimentally has an isoelectric point of 6.89 and should thus be negatively charged at pH 7.2, while the calculated pH titration shows it has a slightly positive charge.

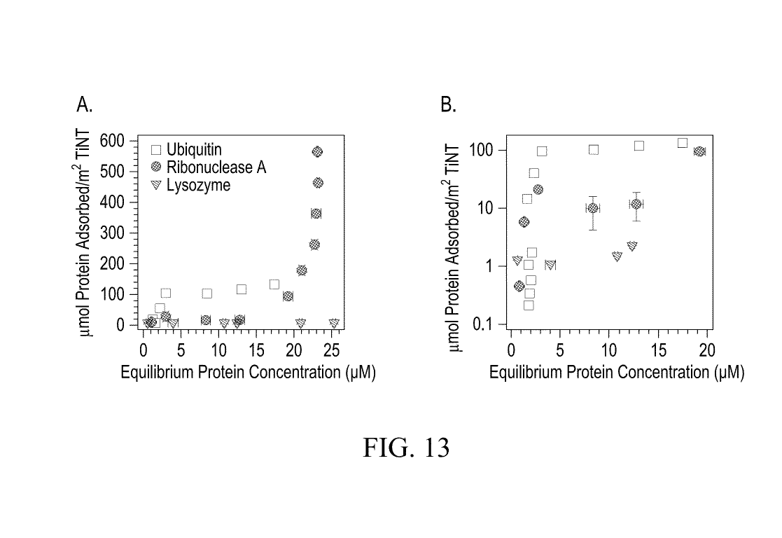

In FIGS. 13A and 13B the equilibrium adsorption isotherms for RNaseA, Lysozyme, and Ubiquitin at pH 7.2 are shown. Although the behavior of RNaseA at c*=23 μM was not exhibited by the other two proteins, the adsorption capacity of all three proteins significantly exceeded monolayer coverage (indicated in Table 1). As seen in FIG. 13A, before c* the amount of protein adsorbed was largest for Ubiqituin, which has nearly neutral charge at pH 7.2, and was smallest for Lysozyme, which has the largest charge. This is consistent with experimental observations that protein adsorption on a substrate is maximized near the protein's isoelectric point due to the decreased protein-protein repulsion.

The differences between the RNaseA and Lysozyme adsorption isotherms may also be due to their differing dipole moments—RNaseA has a large dipole moment, while Lysozyme's is quite small. Due to RNaseA's large dipole it is likely to adsorb in a preferred orientation, while Lysozyme will be more likely to approach the surface of the nanotube with a near-random orientation with a significantly less efficient packing density.

The nanotube and RNaseA are oppositely charged at pH 7.2 and experience a net Coulombic attraction to each other. Therefore, to investigate the role of electrostatic interactions between the protein and nanotube, a series of trials was performed with a fixed concentration of nanotubes and RNaseA and varying amounts of NaCl and examined the mean aggregate size using dynamic light scattering (DLS).

The results of these experiments demonstrate significant aggregation of all three proteins tested (RNaseA, Lysozyme and Ubq), and evidences that similar aggregation can be expected from other globular proteins and biomolecules.

### Example 11

### Pickering Emulsions of Cryomilled Nanotubes

TiNT can have numerous applications in petrophysical applications, including use as a dielectric contrast agent. In investigating some of possible applications, studies were conducted to evaluate whether cryomilled TiNT would migrate to or aggregate at an oil-water interface.

Kerosene or toluene was added to a dilute suspension of cryomilled TiNT.

After briefly agitating the solution, it was discovered that a large amount of nanotubes appeared to transfer to the oil phase. This was not observed in control samples. A stable water-in-oil (w/o) emulsion formed in the oil phase and contained 50 μm to 70 μm wide water droplets in the oil phase. Deemulsification occurred slowly and after 1 year a majority of the emulsion remained intact. The large droplet size (>1 μm), high volume fraction of the disperse phase, and metastability are characteristic of macromelusions.

Oil and water are immiscible due to the high surface tension difference between them. Agitation can briefly form a dispersion, but once agitation is removed, the oil and water individually coalesce to reduce their total interfacial area and leads to complete phase separation. A continuous oil phase with observed with a disperse water phase. These emulsions only formed in the presence of the cryomilled nanotubes. Particle stabilized emulsions, called Pickering emulsions, can form when interfacial tension between the particle and each of the individual immiscible liquid phases is smaller than the interfacial tension between the two different liquid phases, as illustrated in FIG. 12.

Adsorption of the nanotube to the oil-water interface eliminates an area of the interface between immiscible phases. Consider a particle of radius, R, which is adsorbed at the oil-water interface. In terms of the contact angle, θ, between particle/water and particle/oil interfaces, the planar area of the oil-water interface that is eliminated by the presence of the particle is:

Aₑ=πR² sin²(θ)=πR²(1−cos²(θ)) (Equation 7)

Assuming R is small enough such that gravity can be neglected, and assuming that the oil-water interface is planar, and designating the surface tension between the different interfaces, γ, with subscripts o(il), w(ater), and p(article). Therefore the energy required to remove a particle from the interface into the oil phase will be:

E=2πR²(1+cos(θ))(γ_{p/o}−γ_{p/w})+πR²(1−cos²(θ))γ_{o/w} (Equation 8)

Relating the surface tensions by the Young-Laplace equation:

γ_{p/o}−γ_{p/w}=γ_{o/w} cos(θ) (Equation 9)

The energy change simplifies to:

$$E = π {R}^{2} {γ}_{o / w} ( 1 + \frac{π {R}^{2}}{{γ}_{o / w}} ( {γ}_{o / w} - {( {γ}_{p / w} - {γ}_{p / o} cos ( θ ) )}^{2} = ) ) \qquad ( Equation 10 )$$

It is clear from the above calculation that if the γ_{p/w}−γ_{p/o}>γ_{o/w}, it will be favorable for the particle to be held at the interface. Of note, the cryomilled nanotubes form stable water oil pickering emulsions over a wide range of pH values.

### Conclusions from Examples 1-11

In summary, the instant disclosure reveals that titania nanotubes with a high density of unterminated undercoordinated Ti surface sites are able to immobilize extraordinarily large quantities of biomolecules, in some instances over 1,000 times above monolayer coverage, while other forms of TiO₂ nanomaterials do not show such properties. This phenomenon has not been reported previously with any other nanomaterial. The instant disclosure shows that biomolecule immobilization and assembly on titania nanotubes can in some embodiments occur in two different stages. For example in the case of RNaseA, at low biomolecule-to-TiO₂ molar ratios, biomolecule immobilization takes place up to approximately 55 layers of coverage. The coverage then remains constant until a critical biomolecule-to-TiO₂ molar ratio is reached. Upon reaching this critical ratio, the system self assembles into large aggregates, above which any subsequently added biomolecules incorporate into the existing self-assembled aggregates. For RNaseA, self assembly occurs at an RNaseA-to-TiO₂ molar ratio of 2 and was observed in independent experiments employing dynamic light scattering, adsorption measurements, and electron microscopy. The self-assembled product is micron-sized, immobilizing as much as 920 g/g RNaseA/TiO₂. Moreover, such self-assembled aggregates completely retain or even enhance the enzymatic activity.

Although the protein did not oligomerize, it is possible that adsorption on the nanotube surface can significantly alter the protein conformation, however the retention and enhancement of enzymatic activity at high molar ratios suggest that conformation of protein bound far from the nanotube surface is minimally perturbed. The instant disclosure highlights the importance of nanomaterial surface chemistry. Specifically, the surface of the titania nanotube contains a very high density of unterminated undercoordinated Ti sites, which are stable against hydroxylation due to the bond strain imposed by nanotube's curvature. When the nanotube's curvature is removed, such as in the case of nanosheets or nanotiles, the high energy undercoordinated surface Ti sites are instantly terminated by hydroxylation, thereby restoring sixfold coordination. These materials, e.g. nanosheets or nanotiles, can only immobilize biomolecules up to monolayer coverage. Here it has been demonstrated that undercoordinated transition metal sites can play a role in biomolecule immobilization or the templating of larger biomolecule structures. Maintaining enzymatic activity and achieving high immobilization capacities have both been major obstacles for enzyme immobilization. The disclosed results suggest that increasing the density of unterminated undercoordinated transition metal surface sites, either synthetically or by careful control of defect chemistry, stands to be a fruitful strategy for creating novel enzyme immobilization substrates and for creating protein-based biomaterials or enzyme biocatalysts.

### REFERENCES

Chen, Q.; Mogilevsky, G.; Wagner, G. W.; Forstater, J.; Kleinhammes, A.; Wu, Y. Active anatase (001)-like surface of hydrothermally synthesized titania nanotubes. Chem. Phys. Lett. 2009, 482 (1), 134-138.
Forstater, J. H.; Kleinhammes, A.; Wu, Y. Self-Assembly of Protein-Based Biomaterials Initiated by Titania Nanotubes. Langmuir 2013 29, 15013-15021
Han, X.; Kuang, Q.; Jin, M.; Xie, Z.; Zheng, L. Synthesis of titania nanosheets with a high percentage of exposed (001) facets and related photocatalytic properties. J. Am. Chem. Soc. 2009, 131 (9), 3152-3153.
Mogilevsky, G.; Chen, Q.; Kulkarni, H.; Kleinhammes, A.; Mullins, W. M.; Wu, Y. Layered Nanostructures of Delaminated Anatase: Nanosheets and Nanotubes. J. Phys. Chem. C 2008(a), 112 (9), 3239-3246.
Mogilevsky, G.; Chen, Q.; Kleinhammes, A.; Wu, Y. The structure of multilayered titania nanotubes based on delaminated anatase. Chem. Phys. Lett. 2008(b), 460 (4-6), 517-520 (b).

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A biomolecule immobilization substrate comprising a titania nanotube, wherein the titania nanotube comprises a surface and stable undercoordinated titanium sites on the surface, wherein the titania nanotube binds biomolecules.

2. The biomolecule immobilization substrate of claim 1, wherein the stable undercoordinated titanium sites on the surface of the titania nanotube bind biomolecules under physiological conditions.

3. The biomolecule immobilization substrate of claim 1, wherein the binding of biomolecules is non-covalent.

4. The biomolecule immobilization substrate of claim 1, wherein the biomolecule is a protein.

5. The biomolecule immobilization substrate of claim 4, wherein the protein is an enzyme.

6. The biomolecule immobilization substrate of claim 5, wherein the bound enzyme has an enzymatic activity substantially similar to an enzymatic activity of an unbound enzyme.

7. The biomolecule immobilization substrate of claim 5, wherein the bound enzyme has an enzymatic activity that is increased as compared to an enzymatic activity of an unbound enzyme.

8. The biomolecule immobilization substrate of claim 1, wherein the biomolecules are bound to the surface of the titania nanotube in multiple layers.

9. The biomolecule immobilization substrate of claim 1, wherein the titania nanotube has a diameter of about 8 nm to about 14 nm, and a length of about 50 nm to about 3000 nm.

10. The biomolecule immobilization substrate of claim 1, wherein the titania nanotube has an isoelectric point of about 2.0 pH to about 3.0 pH.

11. A method of immobilizing a biomolecule, the method comprising:
providing a titania nanotube comprising a surface with stable undercoordinated titanium sites on the surface;
providing a biomolecule to be immobilized; and
exposing the biomolecule to the titania nanotube;
whereby the biomolecule is immobilized on the surface of the titania nanotube.

12. The method of claim 11, wherein the stable undercoordinated titanium sites on the surface of the titania nanotube bind biomolecules under physiological conditions.

13. The method of claim 12, wherein the binding of biomolecules is non-covalent.

14. The method of claim 11, wherein the biomolecule is a protein.

15. The method of claim 14, wherein the protein is an enzyme.

16. The method of claim 15, wherein the bound enzyme has an enzymatic activity substantially similar to an enzymatic activity of an unbound enzyme.

17. The method of claim 15, wherein the bound enzyme has an enzymatic activity that is increased as compared to an enzymatic activity of an unbound enzyme.

18. The method of claim 11, wherein the titania nanotube has a diameter of about 8 nm to about 14 nm, and a length of about 50 nm to about 3000 nm.

19. The method of claim 11, wherein the titania nanotube has an isoelectric point of about 2.0 pH to about 3.0 pH.

20. The method of claim 11, further comprising exposing a plurality of biomolecules to the titania nanotube.

21. The method of claim 20, wherein the biomolecules are bound to the surface of the titania nanotube in multiple layers.

22. The method of claim 20, wherein exposing a plurality of biomolecules to the titania nanotube results in the self-organized formation of biomolecule-nanotube conjugates.

23. The method of claim 11, wherein the biomolecule substantially maintains its original confirmation.

24. The method of claim 11, wherein exposing the biomolecule to the titania nanotube comprises combining a biomolecule and a titania nanotube into a solution.

25. A method for storing a biomolecule, the method comprising:
providing a titania nanotube comprising a surface and stable undercoordinated titanium sites on the surface;
providing a biomolecule to be stored; and
mixing the biomolecule and the titania nanotube in a solution, wherein the solution is a physiological solution having a pH ranging from about 6 to about 8,
whereby the biomolecule is immobilized on the surface of the titania nanotube and is stable for storage.

26. The method of claim 25, further comprising mixing a plurality of biomolecules with a plurality of titania nanotubes.

27. The method of claim 26, wherein a quantity of biomolecules is mixed with a quantity of titania nanotubes sufficient to form a monolayer of biomolecules on the nanotubes.

28. The method of claim 26, wherein a quantity of biomolecules is mixed with a quantity of titania nanotubes sufficient to form a multilayer of biomolecules on the nanotubes.

29. The method of claim 26, wherein a quantity of biomolecules is mixed with a quantity of titania nanotubes which results in the self-organized formation of biomolecule-nanotube aggregates.

30. The method of claim 29, wherein the biomolecule-nanotube aggregates are about one micron in size.

31. A method for enhancing an enzymatic activity of a biomolecule, the method comprising:
providing a titania nanotube comprising a surface and stable undercoordinated titanium sites on the surface;
providing a biomolecule with an enzymatic activity; and
mixing the biomolecule and the titania nanotube in a solution, whereby the biomolecule non-covalently binds to the titania nanotube,
whereby the enzymatic activity of the biomolecule is increased above that of a biomolecule with enzymatic activity that is not bound to a titania nanotube.

32. The method of claim 31, further comprising mixing a plurality of biomolecules with a plurality titania nanotubes.

33. The method of claim 32, wherein a quantity of biomolecules is mixed with a quantity of titania nanotubes sufficient to form a monolayer of biomolecules on the nanotubes.

34. The method of claim 32, wherein a quantity of biomolecules is mixed with a quantity of titania nanotubes sufficient to form a multilayer of biomolecules on the nanotubes.

35. The method of claim 32, wherein a quantity of biomolecules is mixed with a quantity of titania nanotubes which results in the self-organized formation of biomolecule-nanotube aggregates.

36. The method of claim 31, wherein the enzymatic activity of the biomolecules bound to the titania nanotubes is increased by about 10% to about 90%.
